(19) United States
(12) Patent Application Publication
(10) Pub. No.: US 2006/0014741 A1
(43) Pub. Date: Jan. 19, 2006

(54) Synthetic process, and crystalline forms of a pyrrolotriazine compound

(76) Inventors: John D. DiMarco, East Brunswick, NJ (US); Jack Z. Gougoutas, Princeton, NJ (US); Bharat P. Patel, Pennington, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/149,525

(22) Filed: Jun. 10, 2005

Related U.S. Application Data
(63) Continuation-in-part of application No. 11/008,719, filed on Dec. 9, 2004.
(60) Provisional application No. 60/529,347, filed on Dec. 12, 2003.

(51) Int. Cl. A61K 31/5377; C07D 487/04

(52) U.S. Cl. 514/234.2; 544/112

(57) ABSTRACT

The present invention provides a process for preparing pyrrolotriazine compounds of formula (I) 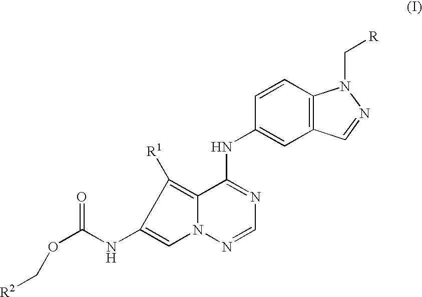 or a pharmaceutically acceptable salt thereof. Also provided are crystalline forms of the pyrrolotriazine compound [4-[[1-(3-fluorophenyl)methyl]-1H-indazol-5-ylamino]-5-methyl-pyrrolo[2, 1-f][1,2,4]triazin-6-yl]-carbamic acid, (3S)-3-morpholinylmethyl ester and pharmaceutical compositions comprising at least one crystalline form, as well of methods of using the crystalline forms in the treatment of a proliferative disease, and methods for obtaining such crystalline forms. The compounds of formula (I), including [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester, are useful for inhibiting tyrosine kinase activity of growth factor receptors such as HER1, HER2 and HER4 thereby making them useful as antiproliferative agents for the treatment of cancer and other diseases.

This application is a continuation-in-part of U.S. patent application Ser. No. 11/008,719, filed Dec. 9, 2004, which claims priority from U.S. Provisional Application No. 60/529,347 filed Dec. 12, 2003, both of which are incorporated herein by reference in their entirety.

### FIELD OF THE INVENTION

This invention relates to novel, improved processes for the preparation of pyrrolotriazine compounds that inhibit the tyrosine kinase activity of growth factor receptors such as HER1, HER2, and HER4 thereby making them useful as anti-cancer agents. The compounds prepared by the processes of the invention are also useful in the treatment of diseases, other than cancer, which are associated with signal transduction pathways operating through growth factor receptors such as HER1, HER2, and HER4.

Also provided are crystalline forms of the pyrrolotriazine compound [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester. The present invention also generally relates to a pharmaceutical composition comprising at least one crystalline form, as well as methods of using the crystalline forms in the treatment of a proliferative disease, such a cancer, and other diseases that are associated with the signal transduction pathways operating through growth factor receptors such as HER1, HER2, and HER4, and methods for obtaining such crystalline forms.

### SUMMARY OF THE INVENTION

The present invention provides an improved process for the preparation of pyrrolotriazine compounds (I) and intermediates (Compounds A and C) for the preparation thereof.

The process of the invention comprises, in one embodiment, the steps of first, chlorinating the pyrrolotriazine core, adding the substituted indazole portion of the compound through an alkylation reaction and then, via Curtius rearrangement, adding the N-protected heterocyclic “tail”. Subsequent deprotection provides the compounds of the invention.

In a second embodiment, the invention provides processes for preparing the key intermediates that are amenable to large scale preparations and provides derivatives of high quality and significantly higher yield than previous processes.

In a third embodiment, the invention provides the N-2 crystalline form of the pyrrolotriazine compound [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

In a fourth embodiment, the invention provides the H-1 monohydrate crystalline form of the pyrrolotriazine compound [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

In a fifth embodiment, the invention provides the N-1 crystalline form of the hydrochloric acid salt of the pyrrolotriazine compound [4-[[i-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

In the sixth embodiment, the invention provides a pharmaceutical composition comprising at least one of the N-2, H-1, or N-1 crystalline forms of the pyrrolotriazine compound [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester; and a pharmaceutically acceptable carrier or diluent.

In the seventh embodiment, the invention provides a method of treating a proliferative disease, such as cancer, comprising administering to a warm blooded animal in need thereof, a therapeutically-effective amount of at least one of the N-2, H-1, or N−1 crystalline forms of the pyrrolotriazine compound [4-[[i-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

The names used herein to characterize a specific form, e.g. “N-1” etc., should not be considered limiting with respect to any other substance possessing similar or identical physical and chemical characteristics, but rather it should be understood that these designations are mere identifiers that should be interpreted according to the characterization information also presented herein.

### BRIEF DESCRIPTION OF THE DRAWINGS

FIG. 1 shows observed and simulated powder x-ray diffraction patterns (CuKα λ=1.5418 Å at T=22-C) of the N-2 crystalline form of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

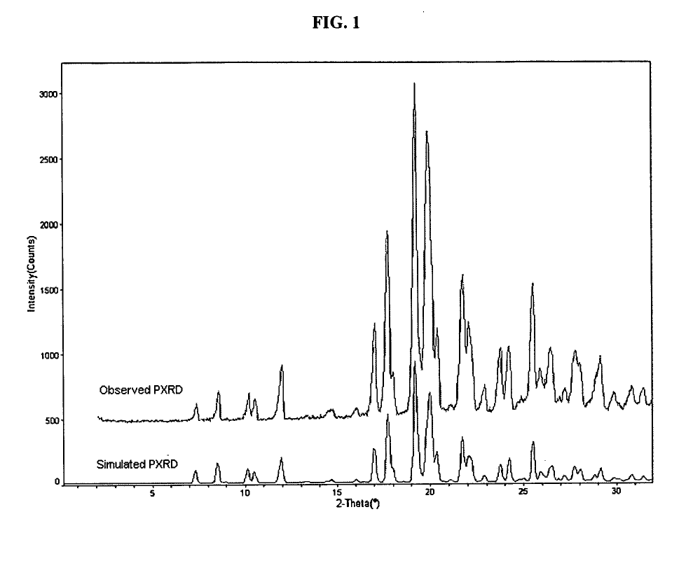

FIG. 2 shows observed and simulated powder x-ray diffraction patterns (CuKα λ=1.5418 Å at T=22° C.) of the H-1 crystalline form of the monohydrate of [4-[[i-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

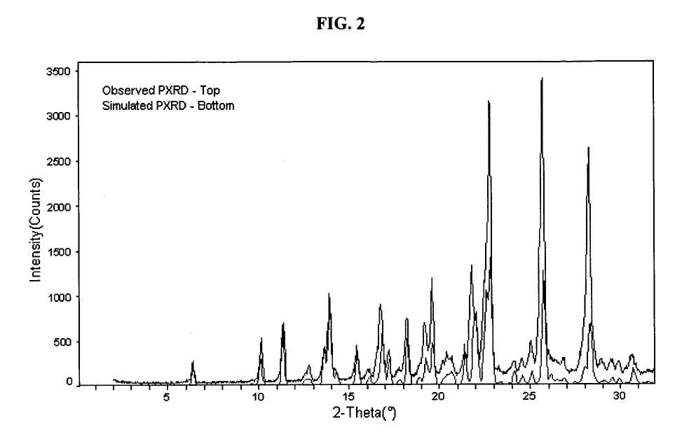

FIG. 3 shows observed and simulated powder x-ray diffraction patterns (CuKα λ=1.5418 Å at T=22° C.) of the N-1 crystalline form of HCl salt of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

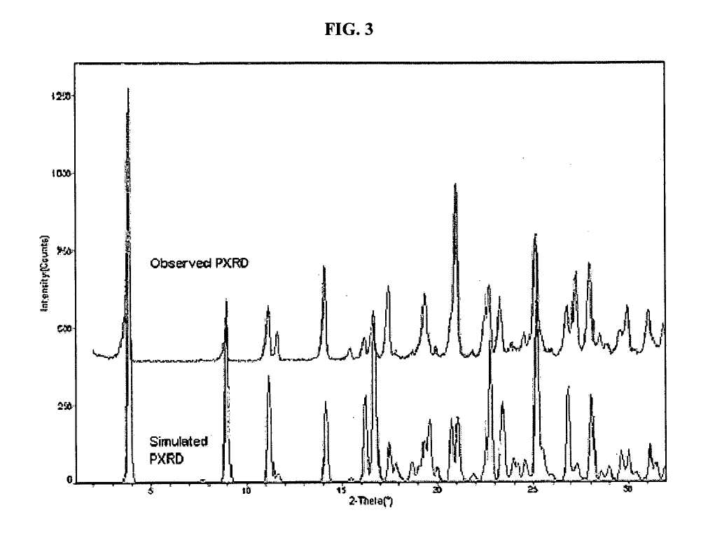

FIG. 4 shows a differential calorimetry thermogram (DSC) of the N-2 crystalline form of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

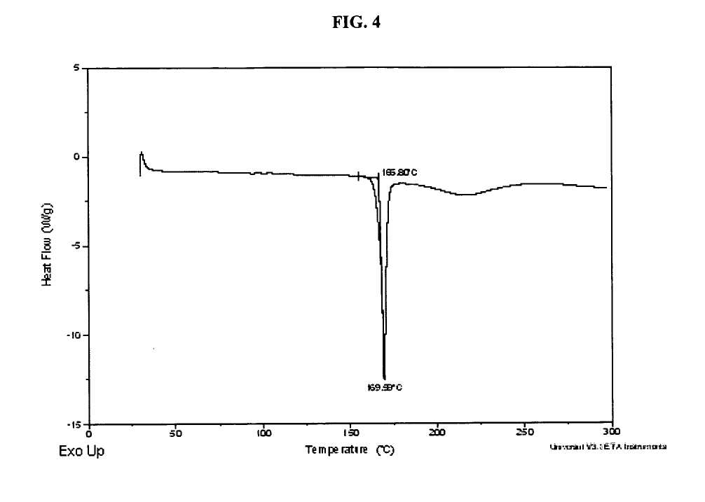

FIG. 5 shows a differential calorimetry thermogram and the thermogravimetric weight loss (TGA) of the H-1 crystalline form of [4-[[i-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

FIG. 6 shows a differential calorimetry thermogram of the N−1 crystalline form of HCl salt of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester.

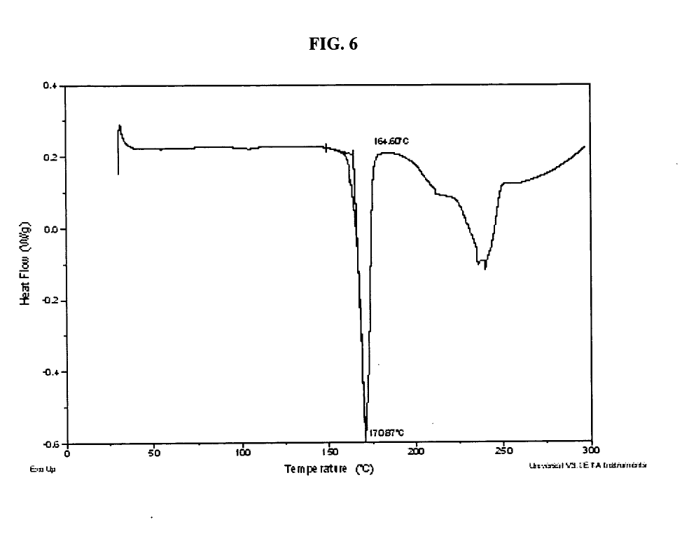

### DETAILED DESCRIPTION OF THE INVENTION

The present invention provides a process for the preparation of compounds of the formula 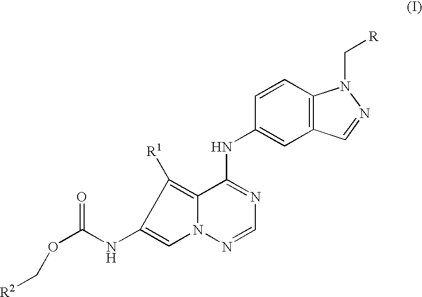
wherein
R is aryl, substituted aryl, heterocyclyl, or substituted heterocyclyl;
R¹ is alkyl or substituted alkyl; and
R² is heterocyclyl or substituted heterocyclyl;
   or a pharmaceutically acceptable salt or stereoisomer thereof,
which comprises the steps of:
   reacting Compound II of the formula or a tautomer thereof 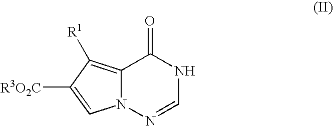
   with an activating agent such as phosphorus oxyhalide or a Vilsmeier Reagent to afford Compound II of the formula,
   wherein
X is a leaving group such as Cl, Br, I or a phosphorus ester,
R¹ is as defined above, and
R³ is lower alkyl 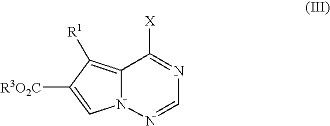
   which is subsequently coupled with Compound IV of the formula 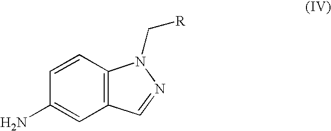
   where R is as defined above,
to afford Compound V of the formula 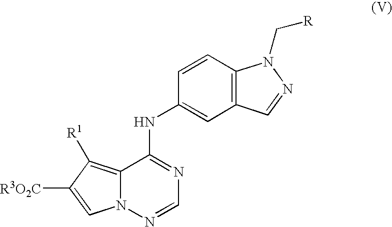
which is hydrolyzed to afford Compound VI of the formula 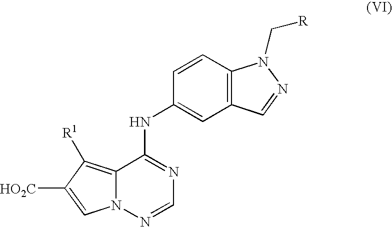
   which subsequently undergoes a Curtius rearrangement in the presence of a compound of the formula R²CH₂OH to afford Compound I.

There is also disclosed a process for preparing the compound of the formula 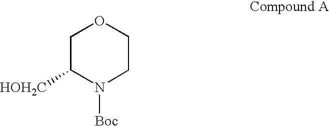
or a stereoisomer thereof, which comprises the steps of:
reacting Compound VII of the formula 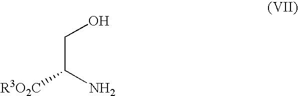
or a salt thereof, wherein R³ is alkyl,
with a substituted or unsubstituted aryl aldehyde in the presence of a reducing agent to afford Compound VIII of the formula 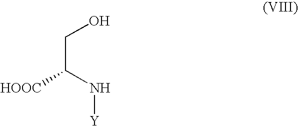
   where Y is benzyl or a substituted benzyl group,
   which is subsequently reacted with an acylating agent in the presence of a mild alkaline buffer to afford Compound IX of the formula 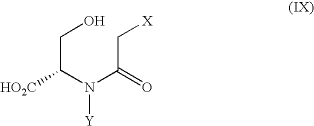
   where X is Cl, Br or I;
   which is cyclized under strongly basic conditions to afford Compound X of the formula 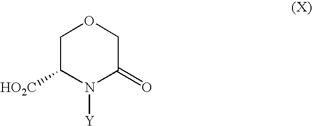
   which is reduced to afford Compound XI of the formula 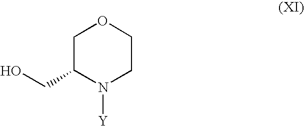
   which is debenzylated and then reacted with a suitable reagent to afford Compound A.

There is also disclosed a process for preparing a compound of the formula 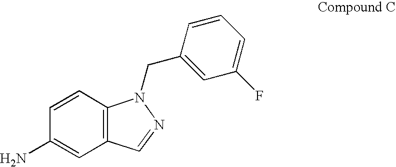
which comprises the steps of
a) alkylating a compound of the formula 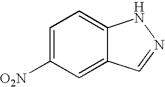
   to afford a compound of the formula 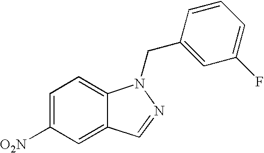
   which is reduced to afford Compound C.

In another embodiment, there is disclosed a process for preparing Compound (Ia) of the formula 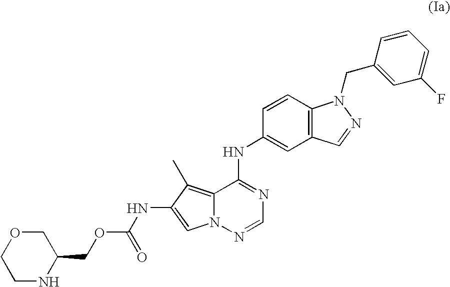
which comprises the steps of:
   reacting Compound B of the formula or a tautomer thereof. 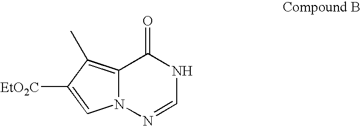
   with an activating agent such as phosphorus oxyhalide, to afford Compound 18 of the formula 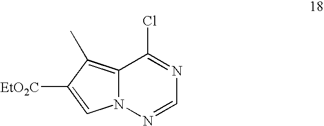
   which is subsequently coupled to Compound C of the formula 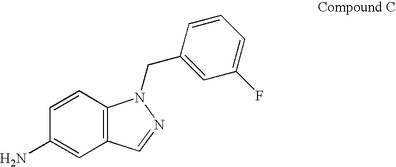
to afford Compound 19 of the formula 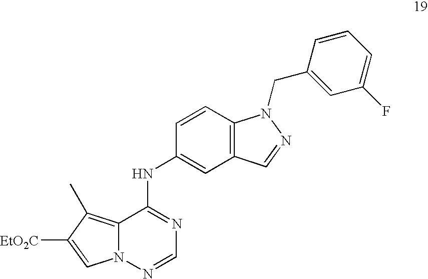
   which is hydrolyzed to afford Compound 20 of the formula 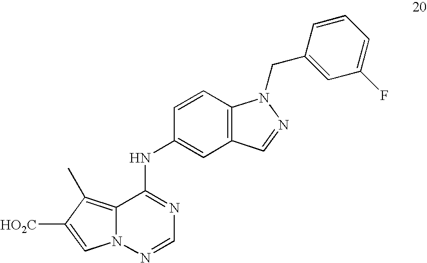
   which subsequently undergoes a Curtius rearrangement in the presence of Compound A, to afford Compound 21 of the formula 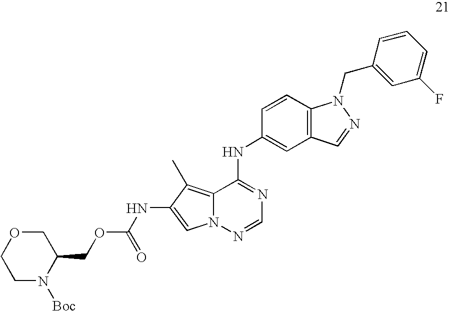
   which is deprotected to afford Compound Ia.

The invention also provides a pharmaceutical composition comprising a Compound of formula I and a pharmaceutically acceptable carrier, prepared by the process of the invention.

The invention also provides a pharmaceutical composition prepared by the process of the invention comprising a Compound of formula I in combination with pharmaceutically acceptable carrier and an anti-cancer or cytotoxic agent. In one embodiment said anti-cancer or cytotoxic agent is selected from the group consisting of linomide; inhibitors of integrin ανβb 3 function; angiostatin; razoxane; tamoxifen; toremifene; raloxifene; droloxifene; iodoxifene; megestrol acetate; anastrozole; letrozole; borazole; exemestane; flutamide; nilutamide; bicalutamide; cyproterone acetate; gosereline acetate; leuprolide; finasteride; metalloproteinase inhibitors; inhibitors of urokinase plasminogen activator receptor function; growth factor antibodies; growth factor receptor antibodies such as Avastin® (bevacizumab) and Erbitux® (cetuximab); tyrosine kinase inhibitors; serine/threonine kinase inhibitors; methotrexate; 5-fluorouracil; purine; adenosine analogues; cytosine arabinoside; doxorubicin; daunomycin; epirubicin; idarubicin; mitomycin-C; dactinomycin; mithramycin; cisplatin; carboplatin; nitrogen mustard; melphalan; chlorambucil; busulphan; cyclophosphamide; ifosfamide nitrosoureas; thiotepa; vincristine; Taxol® (paclitaxel); Taxotere® (docetaxel); epothilone analogs; discodermolide analogs; eleutherobin analogs; etoposide; teniposide; amsacrine; topotecan; irinotecan, flavopyridols; biological response modifiers and proteasome inhibitors such as Velcade® (bortezomib).

The present invention also relates to crystalline forms of Compound Ia, which are described and characterized herein.

The following are definitions of terms that may be used in the present specification. The initial definition provided for a group or term herein applies to that group or term throughout the present specification individually or as part of another group, unless otherwise indicated.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms.

The term “substituted alkyl” refers to an alkyl group substituted by, for example, one to four substituents, such as, halo, hydroxy, alkoxy, oxo, alkanoyl, aryloxy, alkanoyloxy, amino, alkylamino, arylamino, aralkylamino, disubstituted amines in which the 2 amino substituents are selected from alkyl, aryl or aralkyl; alkanoylamino, aroylamino, aralkanoylamino, substituted alkanoylamino, substituted arylamino, substituted aralkanoylamino, thiol, alkylthio, arylthio, aralkylthio, alkylthiono, arylthiono, aralkylthiono, alkylsulfonyl, arylsulfonyl, aralkylsulfonyl, sulfonamido, e.g. SO₂NH₂, substituted sulfonamido, nitro, cyano, carboxy, carbamyl, e.g. CONH₂, substituted carbamyl e.g. CONHalkyl, CONHaryl, CONHaralkyl or cases where there are two substituents on the nitrogen selected from alkyl, aryl or aralkyl; alkoxycarbonyl, aryl, substituted aryl, guanidino and heterocyclic groups, such as, indolyl, imidazolyl, furyl, thienyl, thiazolyl, pyrrolidyl, pyridyl, pyrimidyl and the like. Where noted above where the substituent is further substituted it will be with alkyl, alkoxy, aryl or aralkyl.

The term “halogen” or “halo” refers to fluorine, chlorine, bromine and iodine.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted.

The term “aralkyl” refers to an aryl group bonded directly through an alkyl group, such as benzyl.

The term “substituted aryl” refers to an aryl group substituted by, for example, one to four substituents such as alkyl, substituted alkyl, halo, trifluoromethoxy, trifluoromethyl, hydroxy, alkoxy, alkanoyl, alkanoyloxy, amino, alkylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. The substituent may be further substituted by hydroxy, alkyl, alkoxy, aryl, substituted aryl, substituted alkyl or aralkyl.

The term “heteroaryl” refers to an optionally substituted, aromatic group for example, which is a 4 to 7 membered monocyclic, 7 to 11 membered bicyclic, or 10 to 15 membered tricyclic ring system, which has at least one heteroatom and at least one carbon atom-containing ring, for example, pyridine, tetrazole, indazole, indole.

The term “alkenyl” refers to straight or branched chain hydrocarbon groups of 2 to 20 carbon atoms, preferably 2 to 15 carbon atoms, and most preferably 2 to 8 carbon atoms, having one to four double bonds.

The term “substituted alkenyl” refers to an alkenyl group substituted by, for example, one to two substituents, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino, indolyl, imidazolyl, furyl, thienyl, thiazolyl, pyrrolidyl, pyridyl, pyrimidyl and the like.

The term “alkynyl” refers to straight or branched chain hydrocarbon groups of 2 to 20 carbon atoms, preferably 2 to 15 carbon atoms, and most preferably 2 to 8 carbon atoms, having one to four triple bonds.

The term “substituted alkynyl” refers to an alkynyl group substituted by, for example, a substituent, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino and heterocyclic groups, e.g. imidazolyl, furyl, thienyl, thiazolyl, pyrrolidyl, pyridyl, pyrimidyl and the like.

The term “cycloalkyl” refers to an optionally substituted, saturated cyclic hydrocarbon ring systems, preferably containing 1 to 3 rings and 3 to 7 carbons per ring which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl, and adamantyl. Exemplary substituents include one or more alkyl groups as described above, or one or more groups described above as alkyl substituents.

The terms “heterocycle”, “heterocyclic” and “heterocyclyl” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized or protected. Examples of this include N-protected morpholine.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiomorpholinyl, thiomorpholinyl sulfoxide, thiomorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic heterocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzimidazolyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, indolyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Exemplary substituents include one or more alkyl or aralkyl groups as described above or one or more groups described above as alkyl substituents.

Also included are smaller heterocyclic groups, such as, epoxides and aziridines.

The term “heteroatoms” shall include oxygen, sulfur and nitrogen.

The Compounds of formula I may form salts which are also within the scope of this invention. Pharmaceutically acceptable (i.e. non-toxic, physiologically acceptable) salts are preferred, although other salts are also useful, e.g., in isolating or purifying the compounds of this invention.

The Compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The Compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

Compounds of formula I may also have prodrug forms. Any compound that will be converted in vivo to provide the bioactive agent (i.e., the compound for formula I) is a prodrug within the scope and spirit of the invention.

Various forms of prodrugs are well known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krosgaard-Larsen and H. Bundgaard, Chapter 5, “Design and Application of Prodrugs,” by H. Bundgaard, p. 113-191 (1991);
c) H. Bundgaard, Advanced Drug Delivery Reviews, 8, 1-38 (1992);

It should further be understood that solvates (e.g., hydrates) of the Compounds of formula I are also within the scope of the present invention. Methods of solvation are generally known in the art.

As used herein “polymorphs” refers to crystalline forms having the same chemical composition but different spatial arrangements of the molecules, and/or ions forming the crystals.

As used herein “solvate” refers to a crystalline form of a molecule and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. Solvent molecules may occur as dimers or oligomers comprising more than one molecule of solvent within the crystalline lattice structure.

As used herein “amorphous” refers to a solid form of a molecule and/or ions that is not crystalline. An amorphous solid does not display a definitive X-ray diffraction pattern with sharp maxima.

As used herein, “substantially pure,” when used in reference to a crystalline form, means a compound having a purity greater than 90 weight %, including greater than 90, 91, 92, 93, 94, 95, 96, 97, 98, and 99 weight %, and also including equal to about 100 weight % of the compound, based on the weight of the compound. The remaining material comprises other form(s) of the compound, and/or reaction impurities and/or processing impurities arising from its preparation. For example, a crystalline form of Compound Ia may be deemed substantially pure in that it has a purity greater than 90 weight % of the crystalline form of Compound Ia, as measured by means that are at this time known and generally accepted in the art, where the remaining less than 10 weight % of material comprises other form(s) of Compound Ia and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

As used herein, the unit cell parameter “molecules/unit cell” refers to the number of molecules of Compound Ia in the unit cell.

The present invention provides, at least in part, crystalline forms of Compound Ia, salts, and solvates thereof. Compound Ia is [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester and has the structure

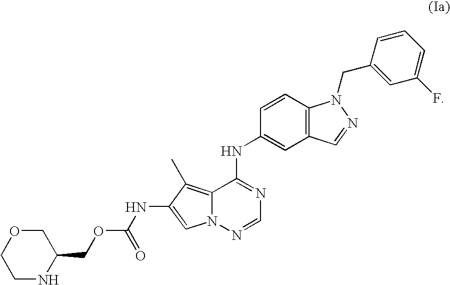

In one aspect of the invention, a crystalline form of the Compound Ia is provided. This crystalline form is a neat crystalline form and is referred to herein as the “N-2” form, which comprises the Compound Ia.

In one embodiment, the N-2 crystalline form may be characterized by unit cell parameters substantially equal to the following:
Cell dimensions: a=10.16 Å
   b=10.46 Å
   c=12.48 Å
   α=96.4 degrees
   β=103.3 degrees
   γ=93.7 degrees
Space group: P1
Molecules/unit cell: 2
Volume: 1277.5 Å³
Density (calculated): 1.379 g/cm³
   wherein measurement of said crystalline form is at a temperature of about 25° C.

In a different embodiment, the N-2 crystalline form may be characterized by a powder x-ray diffraction pattern comprising four or more 2θ values (CuKαλ=1.5418 Å), preferably five or more 2θ values, selected from the group consisting of 7.3, 8.6, 12.0, 17.8, 19.3, 20.1, and 25.6, at a temperature of 22° C.

In another aspect of the invention, a different crystalline form of the Compound Ia is provided. This crystalline form is a monohydrate crystal comprising Compound Ia and water and is referred to herein as the “H-1” form.

In one embodiment, the H-1 crystalline form may be characterized by unit cell parameters substantially equal to the following:
Cell dimensions: a=8.78 Å
   b=10.78 Å
   c=14.08 Å
   α=99.6 degrees
   β=95.8 degrees
   λ=93.3 degrees
Space group: P1
Molecules/unit cell: 2
Volume: 1303.9 Å³
Density (calculated): 1.397 g/cm³
   wherein measurement of said crystalline form is at a temperature of about 25° C.

In a different embodiment, the H-1 crystalline form may be characterized by a powder x-ray diffraction pattern comprising four or more 2θ values (CuKαγ=1.5418 Å), preferably five or more 2θ values, selected from the group consisting of 6.5, 10.2, 11.4, 15.5, 18.3, 22.9, 25.8, and 28.4, at a temperature of 22° C.

In a still different aspect of the invention, a crystalline form of the hydrochloric acid salt of Compound Ia is provided. This crystalline form is a salt formed between hydrochloric acid and Compound Ia and is referred to herein as the “N-1” form.

In one embodiment, the N−1 crystalline form may be characterized by unit cell parameters substantially equal to the following:
Cell dimensions: a=5.32 Å
   b=10.92 Å
   c=22.95 Å
   α=90.0 degrees
   β=94.9 degrees
   λ=90.0 degrees
Space group: P2₁
Molecules/unit cell: 2
Volume: 1327.6 Å³
Density (calculated): 1.418 g/cm³
   wherein measurement of said crystalline form is at a temperature of about 25° C.

In a different embodiment, the N−1 crystalline form may be characterized by a powder x-ray diffraction pattern comprising four or more 2θ values (CuKαλ=1.5418 Å), preferably five or more 2θ values, selected from the group consisting of 3.9, 9.0, 11.3, 14.2, 16.8, 25.3, and 26.9, at a temperature of 22° C.

In one embodiment of the invention, a crystalline form of the Compound Ia, for example, the N-1, N-2, or H-1 form, is provided in substantially pure form. This crystalline form of Compound Ia in substantially pure form may be employed in pharmaceutical compositions which may optionally include one or more other components selected, for example, from the group consisting of excipients, carriers, and one of other active pharmaceutical ingredients active chemical entities of different molecular structure.

Preferably, the crystalline form has substantially pure phase homogeneity as indicated by less than 10%, preferably less than 5%, and more preferably less than 2% of the total peak area in the experimentally measured PXRD pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

In one embodiment, a composition is provided consisting essentially of the crystalline form N-2 of the Compound Ia. The composition of this embodiment may comprise at least 90 weight % of the crystalline form N-2 of Compound Ia, based on the weight of Compound Ia in the composition.

In a different embodiment, a composition is provided consisting essentially of the crystalline form H-1 of the Compound Ia. The composition of this embodiment may comprise at least 90 weight % of the crystalline form H-1 of Compound Ia, based on the weight of Compound Ia in the composition.

In a still different embodiment, a composition is provided consisting essentially of the crystalline form N-1 of the Compound Ia. The composition of this embodiment may comprise at least 90 weight % of the crystalline form N-1 of Compound Ia, based on the weight of Compound Ia in the composition.

### Use and Utility

Pyrrolotriazine compounds of formula I, such as Compound Ia, inhibit the protein tyrosine kinase activity of members of the HER family of receptors. These inhibitors will be useful in the treatment of proliferative diseases, such as those that are dependent on signaling by one or more of these receptors. Such diseases include psoriasis, rheumatoid arthritis, and solid tumors of the lung, head and neck, breast, colon, ovary, and prostate. The compound may be administered as a pharmaceutical composition comprising the pyrrolotriazine compound of formula I, or pharmaceutically acceptable salt or hydrate thereof, and a pharmaceutically acceptable carrier. The pyrrolotriazine compounds are useful for treating hyperproliferative disorders in mammals. In particular, the pharmaceutical composition is expected to inhibit the growth of those primary and recurrent solid tumors which are associated with HER1 (EGF receptor) and HER2, especially those tumors which are significantly dependent on HER1 or HER2 for their growth and spread, including for example, cancers of the bladder, squamous cell, head, colorectal, esophageal, gynecological (such as ovarian), pancreas, breast, prostate, vulva, skin, brain, genitourinary tract, lymphatic system (such as thyroid), stomach, larynx, and lung. In another embodiment, the pyrrolotriazine compounds of formula I are also useful in the treatment of noncancerous disorders such as psoriasis and rheumatoid arthritis. A preferred pyrrolotriazine compound of formula I is the pyrrolotriazine compound of formula Ia. More preferably, the pyrrolotriazine compound of formula Ia is provided in the crystalline form N-2.

Thus according to a further aspect of the invention there is provided the use of a compound of formula Ia, or a pharmaceutically acceptable salt thereof in the manufacture of a medicament for use in the production of an antiproliferative effect in a warm-blooded animal such as a human being. Preferably, the medicament comprises the crystalline form N-2, H-1, or N−1 (HCl salt) of the compound of formula Ia. More preferably, the medicament comprises the N-2 crystalline form of the compound of formula Ia.

According to a further feature of the invention there is provided a method for producing an antiproliferative effect in a warm-blooded animal, such as a human being, in need of such treatment which comprises administering to said animal an effective amount of a pyrrolotriazine compound of formula I or a pharmaceutically acceptable salt thereof as defined herein before.

By virtue of their ability to inhibit HER1, HER2 and HER4 kinases, the pyrrolotriazine compounds of formula I can be used for the treatment of proliferative diseases, including psoriasis and cancer. The HER1 receptor kinase has been shown to be expressed and activated in many solid tumors including head and neck, prostate, non-small cell lung, colorectal, and breast cancer. Similarly, the HER2 receptor kinase has been shown to be overexpressed in breast, ovarian, lung and gastric cancer. Monoclonal antibodies that downregulate the abundance of the HER2 receptor or inhibit signaling by the HER1 receptor have shown anti-tumor efficacy in preclinical and clinical studies. It is therefore expected that inhibitors of the HER1 and HER2 kinases will have efficacy in the treatment of tumors that depend on signaling from either of the two receptors. In addition, these compounds will have efficacy in inhibiting tumors that rely on HER receptor heterodimer signaling. These compounds are expected to have efficacy either as single agent or in combination (simultaneous or sequentially) with other chemotherapeutic agents such as Taxol, adriamycin, and cisplatin. Since HER1 and HER2 signaling has been shown to regulate expression of angiogenic factors such as vascular endothelial growth factor (VEGF) and interleukin 8 (IL8), these compounds are expected to have anti-tumor efficacy resulting from the inhibition of angiogenesis in addition to the inhibition of tumor cell proliferation and survival. The HER2 receptor has been shown to be involved in the hyperproliferation of synovial cells in rheumatoid arthritis, and may contribute to the angiogenic component of that inflammatory disease state. The inhibitors described in this invention are therefore expected to have efficacy in the treatment of rheumatoid arthritis. The ability of these compounds to inhibit HER1 further adds to their use as anti-angiogenic agents. See the following documents and references cited therein: Schlessinger J., “Cell signaling by receptor tyrosine kinases”, Cell 103(2), p. 211-225 (2000); Cobleigh, M. A., Vogel, C. L., Tripathy, D., Robert, N. J., Scholl, S., Fehrenbacher, L., Wolter, J. M., Paton, V., Shak, S., Lieberman, G., and Slamon, D. J., “Multinational study of the efficacy and safety of humanized anti-HER2 monoclonal antibody in women who have HER2-overexpressing metastatic breast cancer that has progressed after chemotherapy for metastatic disease”, J. of Clin. Oncol. 17(9), p. 2639-2648 (1999); Baselga, J., Pfister, D., Cooper, M. R., Cohen, R., Burtness, B., Bos, M., D'Andrea, G., Seidman, A., Norton, L., Gunnett, K., Falcey, J., Anderson, V., Waksal, H., and Mendelsohn, J., “Phase I studies of anti-epidermal growth factor receptor chimeric antibody C225 alone and in combination with cisplatin”, J. Clin. Oncol. 18(4), p. 904-914 (2000); Satoh, K., Kikuchi, S., Sekimata, M., Kabuyama, Y., Homma, M. K., and Homma Y., “Involvement of ErbB-2 in rheumatoid synovial cell growth”, Arthritis Rheum. 44(2), p. 260-265 (2001).

The antiproliferative treatment defined herein before may be applied as a sole therapy or may involve, in addition to a pyrrolotriazine compound of formula I, one or more other substances and/or treatments. Such conjoint treatment may be achieved by way of the simultaneous, sequential or separate administration of the individual components of the treatment. The pyrrolotriazine compounds of formula I may also be useful in combination with known anti-cancer and cytotoxic agents and treatments, including radiation. If formulated as a fixed dose, such combination products employ the pyrrolotriazine compounds of formula I within the dosage range described below and the other pharmaceutically active agent within its approved dosage range. The pyrrolotriazine compounds of formula I may be used sequentially with known anticancer or cytotoxic agents and treatment, including radiation when a combination formulation is inappropriate.

In the field of medical oncology it is normal practice to use a combination of different forms of treatment to treat each patient with cancer. In medical oncology the other component(s) of such conjoint treatment in addition to the antiproliferative treatment defined herein before may be: surgery, radiotherapy or chemotherapy. Such chemotherapy may cover three main categories of therapeutic agent: antiangiogenic agents that work by different mechanisms from those defined hereinbefore (for example, linomide, inhibitors of integrin (ανβ3 function, angiostatin, razoxane); cytostatic agents such as antiestrogens (for example, tamoxifen, toremifene, raloxifene, droloxifene, iodoxifene), progestogens (for example, megestrol acetate), aromatase inhibitors (for example, anastrozole, letrozole, borazole, exemestane), antihormones, antiprogestogens, antiandrogens (for example, flutamide, nilutamide, bicalutamide, cyproterone acetate), LHRH agonists and antagonists (for example, gosereline acetate, leuprolide), inhibitors of testosterone 5α-dihydroreductase (for example, finasteride), farnesyltransferase inhibitors, anti-invasion agents (for example, metalloproteinase inhibitors such as marimastat and inhibitors of urokinase plasminogen activator receptor function) and inhibitors of growth factor function, (such growth factors include for example, EGF, FGF, platelet derived growth factor and hepatocyte growth factor, such inhibitors include growth factor antibodies, growth factor receptor antibodies such as Avastin® (bevacizumab) and Erbitux® (cetuximab); tyrosine kinase inhibitors, serine/threonine kinase inhibitors and inhibitors of insulin growth receptor); and antiproliferative/antineoplastic drugs and combinations thereof, as used in medical oncology, such as antimetabolites (for example, antifolates such as methotrexate, fluoropyrimidines such as 5-fluorouracil, purine and adenosine analogues, cytosine arabinoside); Intercalating antitumour antibiotics (for example, anthracyclines such as doxorubicin, daunomycin, epirubicin and idarubicin, mitomycin-C, dactinomycin, mithramycin); platinum derivatives (for example, cisplatin, carboplatin); alkylating agents (for example, nitrogen mustard, melphalan, chlorambucil, busulphan, cyclophosphamide, ifosfamide nitrosoureas, thiotepa; antimitotic agents (for example, vinca alkaloids like vincristine, vinorelbine, vinblastine and vinflunine, and taxoids such as Taxol® (paclitaxel), Taxotere® (docetaxel) and newer microbtubule agents such as epothilone analogs, discodermolide analogs, and eleutherobin analogs); topoisomerase inhibitors (for example, epipodophyllotoxins such as etoposide and teniposide, amsacrine, topotecan, irinotecan); cell cycle inhibitors (for example, flavopyridols); biological response modifiers and proteasome inhibitors such as Velcade® (bortezomib).

As stated above, the pyrrolotriazine compounds of formula I are of interest for their antiproliferative effects. Such compounds are expected to be useful in a wide range of disease states including cancer, psoriasis, and rheumatoid arthritis.

More specifically, the compounds of formula I are useful in the treatment of a variety of cancers, including (but not limited to) the following:
carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma;
tumors of mesenchymal origin, including fibrosarcoma and rhabdomyosarcoma;
tumors of the central and peripheral nervous system, including astrocytoma, neuroblastoma, glioma and schwannomas; and
other tumors, including melanoma, seminoma, teratocarcinoma, and osteosarcoma.

Due to the key role of kinases in the regulation of cellular proliferation in general, inhibitors could act as reversible cytostatic agents, which may be useful in the treatment of any disease process that features abnormal cellular proliferation, e.g., benign prostate hyperplasia, familial adenomatosis polyposis, neuro-fibromatosis, pulmonary fibrosis, arthritis, psoriasis, glomerulonephritis, restenosis following angioplasty or vascular surgery, hypertrophic scar formation and inflammatory bowel disease

The pyrrolotriazine compounds of formula I, including pyrrolotriazine compound of formula Ia, are especially useful in treatment of tumors having a high incidence of tyrosine kinase activity, such as colon, lung, and pancreatic tumors. By the administration of a composition (or a combination) comprising the pyrrolotriazine compounds of formula I, development of tumors in a mammalian host is reduced. The pyrrolotriazine compounds of formula I may also be useful in the treatment of diseases other than cancer that may be associated with signal transduction pathways operating through growth factor receptors such as HER1 (EGF receptor), HER2, or HER4.

The pharmaceutical compositions of the present invention containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose or cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisole or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS.™. model 5400 intravenous pump.

The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the Compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

If formulated as a fixed dose, such combination products employ the compounds of this invention within the dosage range described above and the other pharmaceutically active agent or treatment within its approved dosage range. Compounds of formula I may also be administered sequentially with known anticancer or cytotoxic agents when a combination formulation is inappropriate. The invention is not limited in the sequence of administration; Compounds of formula I may be administered either prior to or after administration of the known anticancer or cytotoxic agent(s).

The compounds may be administered in a dosage range of about 0.05 to about 200 mg/kg/day, preferably less than 100 mg/kg/day, in a single dose or in 2 to 4 divided doses.

In one embodiment, a pharmaceutical composition is provided comprising Compound Ia in crystalline form N-2, H-1, or N-1 (HCl salt), and a pharmaceutically acceptable carrier or diluent. The crystalline form N-2 is preferred. A pharmaceutical composition comprising the N-2 form may be provided with a combination of chemical and/or physical stability to allow preparation of dosage forms with acceptable uniformity and/or storage stability. The N-2 form is not susceptible to the loss of moisture and conversion to a different form.

### Methods of Preparation

Compounds of formula I may be prepared according to the following schemes and the knowledge of one skilled in the art.

All temperatures are in degrees Celsius (° C.) unless otherwise indicated. Preparative Reverse Phase (RP)HPLC purifications were done on C18 reverse phase (RP) columns YMC S5 ODS columns eluting with 90% aqueous methanol containing 0.1% TFA as buffer solution and monitoring at 220 nm. For analytical HPLC 0.2% phosphoric acid was used instead of TFA. All of the synthesized compounds were characterized by at least proton NMR and LC/MS. During work up of reactions, the organic extract was dried over magnesium sulfate (MgSO₄), unless mentioned otherwise.

The following abbreviations are used for the commonly used reagents. Et₂O; diethyl ether, Na₂SO₄; sodium sulfate; HCl; hydrochloric acid, NaOH; sodium hydroxide, NaCl; sodium chloride, Pd/C; palladium on carbon, K₂HPO₄; potassium monohydrogen phosphate, K₂CO₃; potassium carbonate, NaHCO₃; sodium bicarbonate, MgSO₄; magnesium sulfate, LiOH; lithium hydroxide, TMSCl, trimethylsilyl chloride, H₂SO₄, sulfuric acid, RT; room temperature, TFA; trifluoroacetic acid, DMF: dimethyl formamide. Other abbreviation are h; hour, L; liter, ml; milliliter.

The term “Vilsmeier Reagent” means either phosgene iminium chloride (Cl₂C═N(CH₃)₂Cl) or (chloromethylene) dimethylammonium chloride (ClCH═N(CH₃)₂Cl).

The term “activating agent” means phosphorus oxyhalide or Vilsmeier Reagent that converts the amide Compound II to Compound III.

One aspect of the invention involves the preparation of two key intermediates, identified as Compounds A and C.

The original synthesis of Compound A was a very low yielding (overall yield ˜8%) 4-step process. This synthesis is shown below:

### Original Synthesis of Compound A

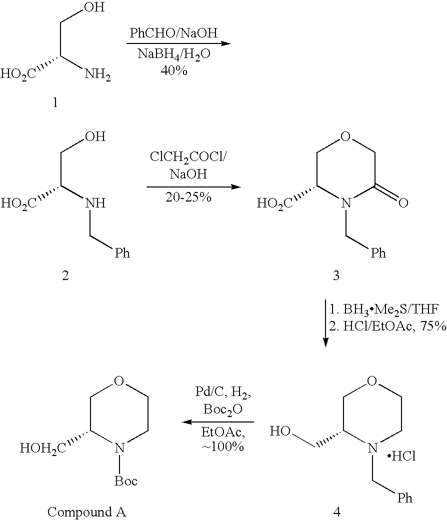 In the original synthesis, the N-benzylation and acylation/cyclization steps were low yielding. This synthesis was based on Brown et al., Journal of the Chemical Society, Perkin Transactions 1: Organic and Bio-Organic Chemistry 1985, 12, pgs 2577-80.

An improved synthesis of this compound was developed using the following steps:

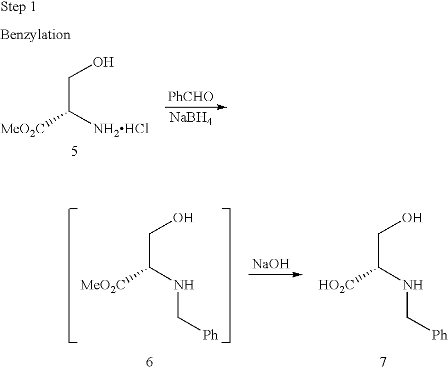

The benzylation step was improved by the use of L-serine methyl ester hydrochloride (5) as the starting material. Other reducing agents used to convert Compound 5 to Compound 7 include catalytic hydrogenation agents and other substituted borohydrides. The yield of this step was increased from the original synthesis from 40% to 60-70%.

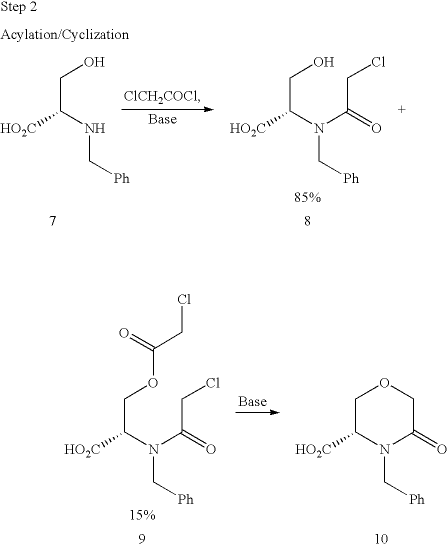

The acylation/cyclization step was improved by carrying out the acylation under Schotten-Baumann conditions using mild bases such as alkali metal carbonates, or alkali metal phosphates like disodium or dipotassium hydrogen phosphate. The cyclization of the acylated material was carried out at lower temperatures (<15° C. compared to the original protocol of 30° C.) and at pH>13. The yield of this step was increased from the original synthesis from 20-25% to 78-85%.

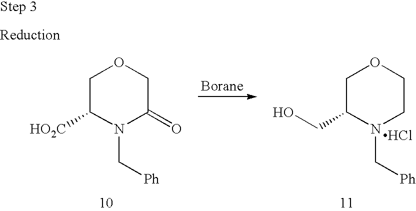

Various reagents were employed in the reduction including BH₃.Me₂S, LAH, Red-Al and lithium triethyl borohydride. Amongst the reagents tried, BH₃.Me₂S provided the best results and was utilized to improve the yield and quality of the desired product. Compound 10 was converted to an alkyl ester and subsequently reduced with LAH to afford Compound 11.

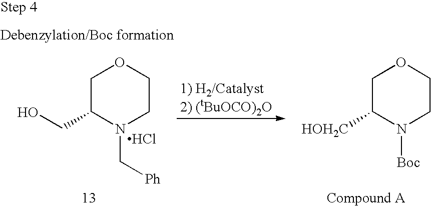

The final step in the process for the preparation of Compound A has been optimized by the use of Pearlman's catalyst (Pd(OH)₂/carbon). The solvent exchange from EtOAc into cyclohexane eliminated concentration to dryness and the product was subsequently crystallized from cyclohexane.

Compound B can be prepared as disclosed in copending application U.S. Ser. No. 10/289,010 published on Oct. 2, 2003 as U.S. 2003/0186982, the disclosure of which is incorporated herein in its entirety.

In the original procedure for the preparation of Compound C, the intermediate Compound 16 in this sequence was obtained in a two step process that involved the alkylation of nitro indazole followed by a recrystallization to obtain the desired N-1 regioisomer. The overall yield of Compound 16 from this two step procedure was only 23%. An improved process for Compound C is shown below where the yield for the preparation of Compound 16 rose to 49%.

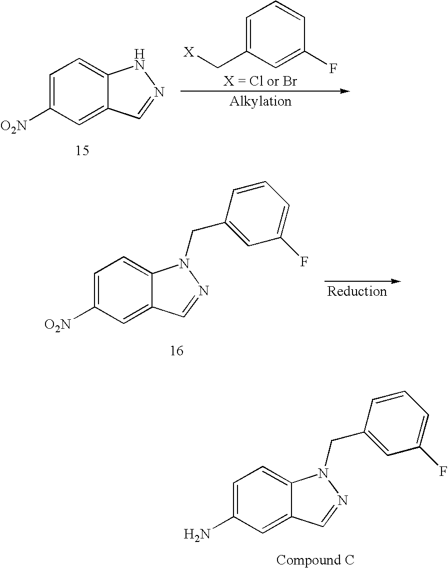

The process is described in more detail below:

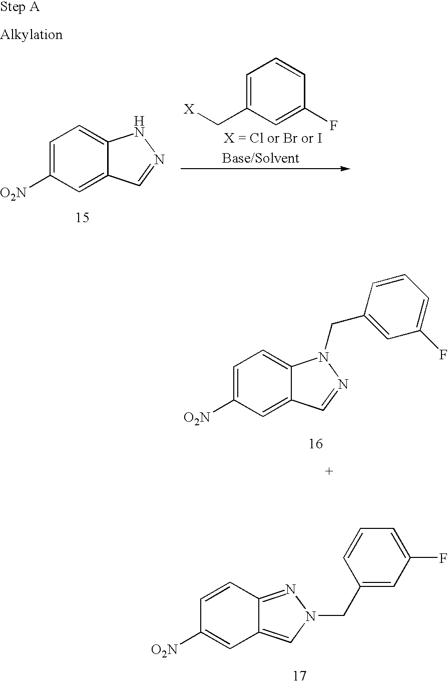

The N-alkylation of the 5-nitroindazole (15) is carried out by treatment with the appropriate alkylating agent in a solvent such as DMF in the presence of a base, such as cesium carbonate and optionally in the presence of a catalyst such as KI. Under these conditions, 70% of the desired isomer (16) and 30% of the undesired regioisomer (17) are obtained. A one pot process has been developed where the desired regioisomer can be crystallized selectively leaving the undesired isomer in solution. The process has been optimized utilizing an electrophile such as aryl bromide, chloride or triflate optionally using KI. This reaction takes place in the presence of a strong alkali base such as Li, Na, or K-HMDS, or alkali carbonates or organolithiums. The choice of solvent is also critical with THF, ACN, DMF, IPA and ethanol being employed.

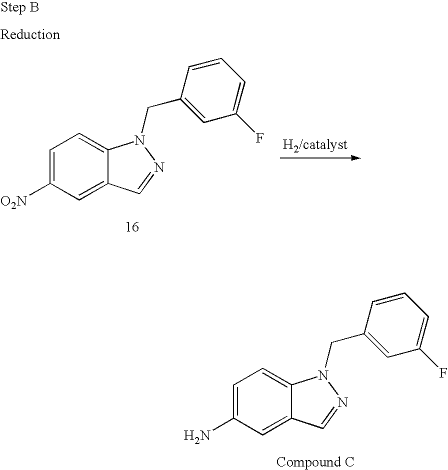

The reduction step incorporates additional process improvements over prior syntheses. These include the use of THF instead of ethanol to prevent crystallization of the product during the reaction and carrying out the hydrogenation at lower pressure (5-15 psi) than in the original process (50 psi).

### Step C

### Chlorination/Alkylation

The final process for conversion of Compound B to Compound 19 via Compound 18 uses 1.8 eq phosphorus oxychloride and 1.2 eq diisopropylethylamine in 15 liters toluene per kilogram (Compound B) at reflux. The reaction was quenched with 6 eq aqueous potassium phosphate dibasic. The rich organic solution of Compound 18 is dried by azeotropic removal of water under reduced pressure to 4 liters per kilogram Compound B final concentration. Additions of Compound C (0.95 eq) and 1.2 eq diisopropylethylamine are followed by warming to 90° C. for 2-4 h. Upon completion, isopropyl alcohol is added to effect crystallization of Compound 19.

Other aqueous quench solutions include water, 1 N hydrogen chloride solution, potassium phosphate tribasic solution, and 1 N sodium hydroxide solution.

Other bases that may be used for the conversion of Compound B to Compound 18 include pyridine.

Use of N-methyl morpholine, DABCO, and pyridine in the conversion of Compound 18 to Compound 19 were evaluated and shown to lead to full or partial conversion.

### Step D

The hydrolysis of the ethyl ester (Compound 19) is carried out using alkali metal hydroxides. The preferred bases are aqueous sodium and potassium hydroxide. This takes place in a combination of hydroxylic and ether solvent at a temperature below 65° C. The carboxylic acid is precipitated from the reaction stream by addition of a mineral acid.

### Step E

A mixture of Compound 20, Compound A, DDPA and an organic tertiary amine is heated at a temperature below 95° C. in an appropriate solvent. The intermediate acyl azide is generated in the presence of Compound A which resulted in minimizing formation of the urea impurity. The Curtius rearrangement can be carried out using polar or nonpolar aprotic solvents such as acetonitrile, toluene or xylene. The complete synthesis of the Compounds of the invention of formula (I) is shown below. 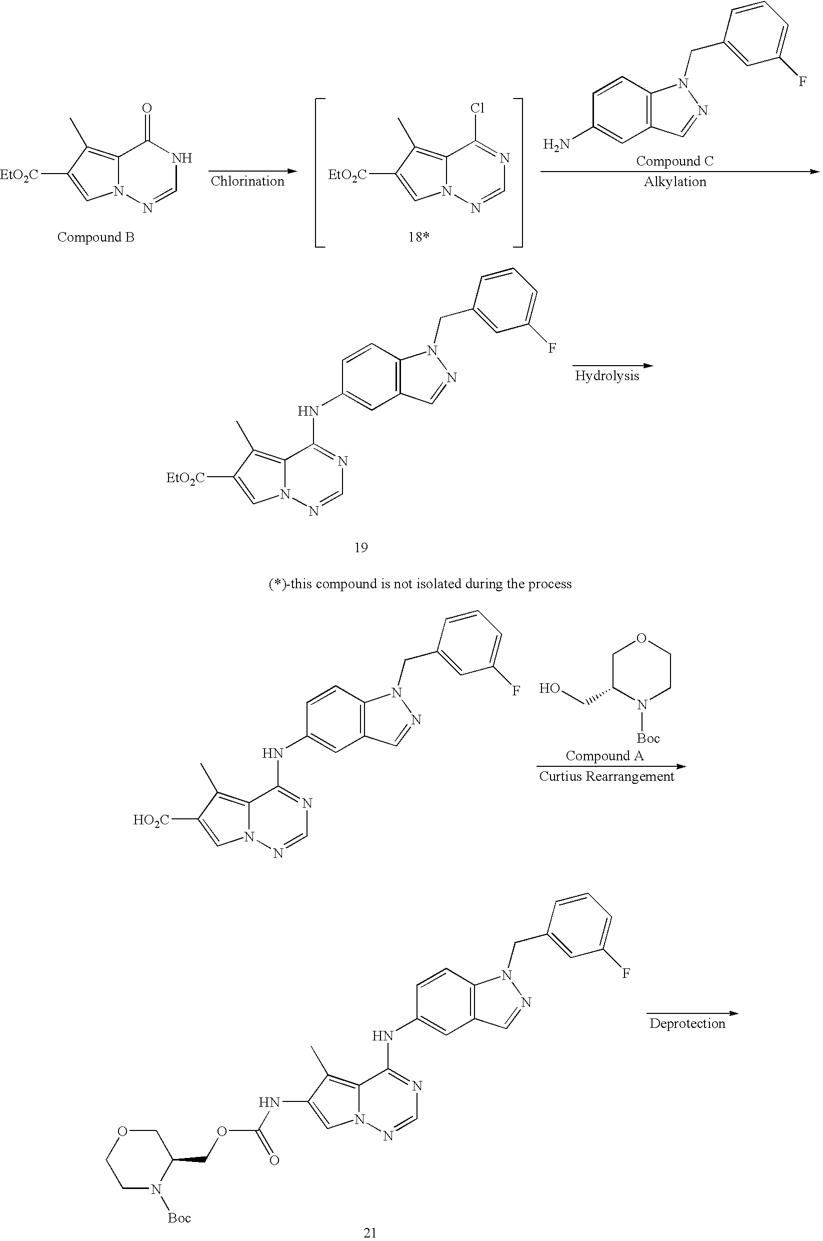 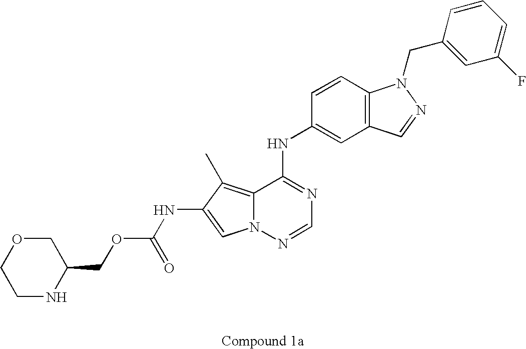
Details of the above process are described below in Example 1.

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Bym, R. R. Pfeiffer, and J. G. Stowell, 2^{nd} Edition, SSCI, West Lafayette, Ind. (1999).

For crystallization techniques that employ solvent, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26,369-377. In general, seeds of small size are needed to control effectively the growth of crystals in the batch. Seed of small size may be generated by sieving, milling, or micronizing of large crystals, or by micro-crystallization of solutions. Care should be taken that milling or micronizing of crystals does not result in any change in crystallinity from the desired crystal form (i.e., change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as solid state nuclear magnetic resonance, differential scanning calorimetry, x-ray powder diffraction, or the like, to assure formation of the preferred crystalline form of the product. The resulting crystalline form may be produced in an amount of greater than about 70 weight % isolated yield, preferably greater than 90 weight % isolated yield, based on the weight of the compound originally employed in the crystallization procedure. The product may be comilled or passed through a mesh screen to delump the product, if necessary.

Crystalline forms may be prepared directly from the reaction medium of the final process for preparing Compound Ia. This may be achieved, for example, by employing in the final process step a solvent or a mixture of solvents from which Compound Ia may be crystallized. Alternatively, crystalline forms may be obtained by distillation or solvent addition techniques. Suitable solvents for this purpose include, for example, the aforementioned nonpolar solvents and polar solvents, including protic polar solvents such as alcohols, and aprotic polar solvents such as ketones.

The presence of more than one crystalline form and/or polymorph in a sample may be determined by techniques such as powder x-ray diffraction (PXRD) or solid state nuclear magnetic resonance spectroscopy. For example, the presence of extra peaks in the comparison of an experimentally measured PXRD pattern with a simulated PXRD pattern may indicate more than one crystalline form and/or polymorph in the sample. The simulated PXRD may be calculated from single crystal x-ray data. see Smith, D. K., “A FORTRAN Program for Calculating X-Ray Powder Diffraction Patterns,” Lawrence Radiation Laboratory, Livermore, Calif., UCRL-7196 (April 1963).

The forms of Compound Ia according to the invention may be characterized using various techniques, the operation of which are well known to those of ordinary skill in the art. The forms may be characterized and distinguished using single crystal x-ray diffraction, which is based on unit cell measurements of a single crystal of form at a fixed analytical temperature. A detailed description of unit cells is provided in Stout & Jensen, X-Ray Structure Determination: A Practical Guide, Macmillan Co., New York (1968), Chapter 3, which is herein incorporated by reference.

Alternatively, the unique arrangement of atoms in spatial relation within the crystalline lattice may be characterized according to the observed fractional atomic coordinates. Another means of characterizing the crystalline structure is by powder x-ray diffraction analysis in which the diffraction profile is compared to a simulated profile representing pure powder material, both run at the same analytical temperature, and measurements for the subject form characterized as a series of 2θ values (usually four or more).

Other means of characterizing the form may be used, such as solid state nuclear magnetic resonance (NMR), differential scanning calorimetry, thermography and gross examination of the crystalline or amorphous morphology. These parameters may also be used in combination to characterize the subject form.

The N-1, N-2, and H-1 crystalline forms may be characterized by single crystal X-ray diffraction measurements performed under standardized operating conditions and temperatures. The approximate unit cell dimensions in Angstroms (Å), as well as the crystalline cell volume, spatial grouping, molecules per cell, and crystal density may be measured, for example at a sample temperature of 25° C.

Each crystalline form was analyzed using one or more of the testing methods described below.

Single Crystal X-Ray Measurements

Single crystal X-ray data for each of Examples 1-3 was collected. For this analysis, a Bruker-Nonius CAD4 serial diffractometer (Bruker Axs, Inc., Madison Wis.); or alternately, a Bruker-Nonius Kappa CCD 2000 system using Cu Kα radiation (λ=1.5418 Å) was used. Unit cell parameters were obtained through least-squares analysis of the experimental diffractometer settings of 25 high-angle reflections. Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Indexing and processing of the measured intensity data were carried out with the HKL2000 software package in the Collect program suite R. Hooft, Nonius B. V. (1998). When indicated, crystals were cooled in the cold stream of an Oxford cryogenic system during data collection.

The structures were solved by direct methods and refined on the basis of observed reflections using either the SDP software package SDP, Structure Determination Package, Enraf-Nonius, Bohemia, N.Y.) with minor local modifications or the crystallographic package, MAXUS (maXus solution and refinement software suit: S. Mackay, C. J. Gilmore, C. Edwards, M. Tremayne, N. Stewart, and K. Shankland. maXus is a computer program for the solution and refinement of crystal structures from diffraction data.

Powder X-Ray Diffraction

X-ray powder diffraction (PXRD) data were obtained using a Bruker GADDS (General Area Detector Diffraction System) manual chi platform goniometer. Powder samples were placed in thin walled glass capillaries of 1 mm or less in diameter; the capillary was rotated during data collection. The sample-detector distance was 17 cm. The radiation was Cu Kα(λ=1.5418 Å). Data were collected for 3<2θ<35° with a sample exposure time of at least 300 seconds.

The derived atomic parameters (coordinates and temperature factors) were refined through full matrix least-squares. The function minimized in the refinements was Σ_{w}(|Fₒ|-|F_{c}|)². R is defined as Σ||F|-|F||/Σ|Fₒ| while R_{w}=[Σ_{w}(|Fₒ|-|F_{c}|)²/Σ_{w}|Fₒ|²]^{1/2} where w is an appropriate weighting function based on errors in the observed intensities. Difference maps were examined at all stages of refinement. Hydrogen atoms were introduced in idealized positions with isotropic temperature factors, but no hydrogen parameters were varied.

Melting Points Melting points for the crystals were determined by hot stage microscopy. Crystals were placed on a glass slide, covered with a cover slip, and heated on a Linkham LTS350 hot stage mounted on a microscope (Linkham Scientific Instruments Ltd, Tadworth, U.K.). The heating rate was controlled at 10° C./min for the temperature range, ambient to 300° C. The crystals were observed visually for evidence of phase transformation, changes in birefringence, opacity, melting, and/or decomposition.

Differential Scanning Calorimetry

Differential scanning calorimetry (DSC) was conducted for each crystalline form using a TA Instruments™ model Q1000. For each analysis, the DSC cell/sample chamber was purged with 100 ml/min of ultra-high purity nitrogen gas. The instrument was calibrated with high purity indium. The heating rate was 10° C. per minute in the temperature range between 25 and 300° C. The heat flow, which was normalized by sample weight, was plotted versus the measured sample temperature. the data were reported in units of watts/gram (“W/g”). The plot was made with the endothermic peaks pointing down. The endothermic melt peak (melting point) was evaluated for extrapolated onset temperature.

The following non-limiting examples are illustrative of the invention.

### EXAMPLE 1

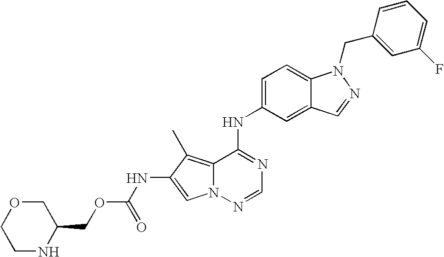

### [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester (Ia)

### A. Preparation of 2-benzylamino-3-hydroxy-propionic acid and 2-dibenzylamino-3-hydroxy-propionic acid

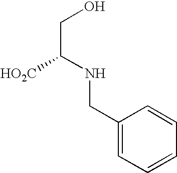

To a reaction vessel were added solid L-serine methyl ester hydrochloride (1.000 equiv.). Methanol (2.85 volumes) was added and agitation was started. Triethylamine (1 equiv.) was added over 10 min while maintaining the temperature from about 14° C. to about 18° C. Stirring was continued until all solids dissolved. The mixture was cooled to 10° C. and benzaldehyde (0.99 equiv.) was added over 15 min while maintaining the temperature between about 11° C. to about 15° C. The reaction was held for 30 min at about 8° C. to about 12° C. Solid sodium borohydride (4 equiv. of hydride) was added over 2 hr while maintaining the temperature at about 10° C. to about 20° C. The reaction was held for 30 min at about 14° C. to about 16° C. and then analyzed by HPLC.

In a separate flask, methanol (1.15 volumes) and water (1.72 volumes) were added. Sodium hydroxide, 50 wt/wt % in water (3.04 equiv.) was added, and the resulting solution was cooled to 15° C. The Schiff's base was transferred to this mixture over 1 hr maintaining the internal temperature between 16-22° C. The reaction was held for 30 min at 20° C. and analyzed by HPLC for consumption of methyl ester. Water (1.72 volumes) was added, followed by concentrated HCl, 12.2 M in water (2.67 equiv.) while maintaining the temperature at 15-25° C. to adjust the pH to 9.5. The mixture was filtered and the filter-cake was washed with two portions of water (0.58 volumes each). The washes were combined with the filtrate in a separatory funnel. The combined aqueous portions were washed two times with ethyl acetate (5.75 volumes each). The material was transferred from the separatory funnel to a flask. The mixture was cooled from 25° C. to 15° C., and concentrated HCl, 12.2 M in water (0.89 equiv.) was added until the pH of the mixture reached 6.5, while maintaining the temperature between 17-22° C. The mixture was held for 15-25 hr at 5° C., then the solids were collected on a filter funnel. The filter cake was washed with two portions of water (1.43 volumes each) and two portions of heptane (1.43 volumes each). The wet solid was transferred to a drying tray, and dried at 45° C. for 21 hr and the yield was 61%.

### B. Preparation of 4-Benzyl-5-oxo-morpholine-3-carboxylic acid

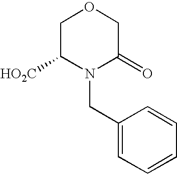

To a reactor was charged N-benzyl-L-serine (1.0 eq) and THF (6.1 volumes). The resulting solution was cooled to 0±5° C. and a pre-cooled solution (0-5° C.) of potassium carbonate (3.0 eq) in water (6.1 volumes) was added. Chloroacetyl chloride (1.4 eq) then was added via addition funnel while maintaining the internal temperature below 5° C. The biphasic reaction mixture was aged for approximately 30 min at 0±5° C. After aging, the mixture was sampled for HPLC analysis. If >6 area percent remaining N-benzyl-L-serine was present, additional chloroacetyl chloride was added. Once the reaction completeness specification has been met, 50 wt % sodium hydroxide is charged while keeping the internal temperature between 5 and 10° C. until the pH remains constant >13.5. The reaction was deemed complete when HPLC analysis showed <1 area percent (combined) intermediates. The mixture was warmed to 25° C., and heptane (2.03 volumes) was added. The mixture was stirred rapidly for 10 min, and then the phases were allowed to separate. The organic upper phase was discarded, and the rich aqueous phase was treated again with heptane (3.04 volumes). After stirring rapidly for 10 min, the phases were allowed to settle, and the organic upper phase was discarded. The rich aqueous portion was cooled to −5 to 0° C. and 37 wt % hydrochloric acid was added while maintaining a batch temperature <10° C. until pH <2. The resulting slurry was kept at −10 to 0° C. for a minimum of 4 h. The slurry was filtered over Whatman 1 filter paper, or equivalent, and washed with pre-cooled (3-7° C.) water (2×4.57 volumes). The wet cake was dried in vacuo at 40-45° C. After drying, 1.475 kg (84.9%, uncorrected) of 4-benzyl-5-oxo-morpholine-3-carboxylic acid was obtained. HPLC Ret Time: 1.82 min (YMC S5 ODS column 4.6×50 mm, 10-90% aqueous methanol over 4 minutes containing 0.2% phosphoric acid, 4 mL/min, monitoring at 220 nm); Chiral HPLC Ret Time: 7.94 min, e.e. 100%, (Chiralcel OJ-R, 150×4.6 mm, 5 μM, eluent: MeOH:0.2% aq. H₃PO4 [50:50], flow rate 1 mL/min, 210 nm)

### C. Preparation of [R-(4-Benzyl-morpholin-3-yl)]-methanol hydrochloride

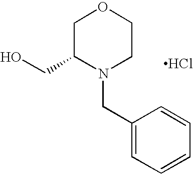

To a stirred mixture of 4-benzyl-5-oxo-morpholine-3-carboxylic acid (1 equiv.) in dry THF (16 volumes) under nitrogen was added triethyl amine (1.19 equiv.). To this mixture was added borane-methyl sulfide complex (7.45 equiv.) at such a rate that the temperature of the reaction mixture was kept below 10° C. The addition took 1 h. The reaction mixture was gently refluxed (65° C.) under nitrogen for 5.5 h. The mixture was cooled and MeOH (1.39 volumes) was added slowly (The internal temperature was kept below 25° C. during the addition and the addition took 1 h). To this resulting mixture was added water (4.18 volumes) and the mixture was stirred at room temperature overnight. The mixture was concentrated in vacuo and was diluted with 2N aqueous sodium hydroxide (4.59 equiv.) and water (1.74 volumes). This mixture was extracted with ethyl acetate (2×7 volumes). The combined ethyl acetate extracts were washed with a 20% aqueous sodium chloride solution (4.18 volumes). The ethyl acetate extracts were then concentrated in vacuo to give a crude oil. This oil was diluted with ethyl acetate (10.2 volumes) and methanol (0.52 volumes). To this solution was added trimethylsilyl chloride (352 mL, 0.61 volumes) dropwise until the pH of the solution was acidic. The batch temperature during the trimethylsilyl chloride addition temperature was kept below 20° C. At the end of the addition, the mixture was cooled at 0° C. for 2 h and the precipitate was collected by filtration to give [R-(4-Benzyl-morpholin-3-yl)]-methanol hydrochloride (547 g) in 92% yield as a white solid.

HPLC: sample preparation: 20 uL in 1 mL caustic for 15 min; AP=98% at 6.19 min (YMC Pack ODS-A, 3 μm column 6.0×150 mm, 10-90% aqueous acetonitrile over 20 minutes containing 0.2% phosphoric acid, 2 mL/min, monitoring at 220 nm and 254 nm)

LC/MS: M+H=208

Chiral HPLC: RT=8.38 min, e.e. 100%, (Chiralcel OD-RH, 150×4.6 mm, eluent: acetonitrile: MeOH:20 mm Ammonium Bicarbonate, pH 7.8 (15:15:70), flow rate 1 mL/min, 210 nM)

### D. Preparation of 3-((R)-Hydroxymethyl)-morpholine-4-carboxylic acid tert-butyl ester

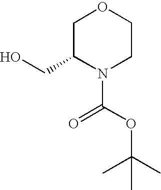

A mixture of [R-(4-benzyl-morpholin-3-yl)]-methanol hydrochloride (1 equiv.), aqueous K₃PO₄ (4.6 equiv), and EtOAc was stirred until two clear phases were obtained. The EtOAc layer was separated, and the aqueous layer was extracted with fresh EtOAc. The combined EtOAc layers were charged into a flask containing 20 wt % Pd(OH)₂/C (50% water wet, 0.10 equiv based on input wt). Di-tert-butyl dicarbonate (1.2 moles) was added. The mixture was hydrogenated for 4 h at 15 psi.

After it was found complete by HPLC, the mixture was filtered through Celite and the solvent was exchanged to cyclohexane. The product was crystallized from cyclohexane (7-10 volumes) to afford the title compound as a white solid (yield 82%).

¹H NMR (CDCl₃) δ 1.45 (s, 9H), 3.17 (m, 1H), 3.47 (dt, 1H, J=3.1, 11.4 Hz), 3.56 (dd, 1H, J=3.5, 11.9 Hz), 3.7-4.0 (m, 6H); ¹³C NMR (CDCl₃) δ 28.21, 40.01, 52.09, 59.59, 65.97, 66.49, 80.23, 155.30; MS: 218 (M+H)⁺; Anal. Calcd for C₁₀H₁₉NO₄: C, 55.28; H, 8.81; N, 6.44. Found: C, 55.45; H, 8.87; N, 6.34; Pd<5 ppm; HPLC Ret Time: 5.28 min (YMC Pack ODS-A, 3 μm, 4.6×50 mm column, 10 min gradient, 2.5 mL/min); 100% ee [Chiral HPLC Ret Time: 13.6 min (Chiralcel OD-RH, 5 μm, 4.6×150 mm column, 20 min wasocratic method, 1 mL/min)].

### E. Preparation of 5-Nitro-1-(3-fluorobenzyl)indazole (16)

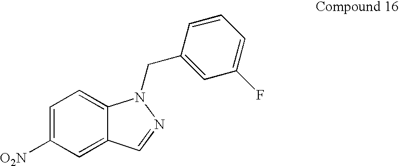

5-nitro indazole (1 equiv.), cesium carbonate (1.1 equiv.) and DMF (5 volumes) were charged to a vessel. The mixture was heated to 70-80° C. and 3-fluoro benzyl bromide was added over 75 mins. The reaction was assayed by HPLC for completion(<2 AP of nitro indazole versus combined isomers) and then cooled to 20° C. The salts were filtered and the cake was washed with DMF (2.7 volumes). The product was crystallized by charging water (1.35 to 1.45 volumes) between 15-21° C. The crystal slurry was held for 4 h, crystals were filtered and washed with 2:1 DMF:water mix (2.1 volumes), water (2 volumes) and finally 3:1 cold ACN:water mix (1.5 volumes). The wet cake was dried <45° C. to LOD<1% and the yield was about 49%.

¹H NMR (CDCl₃) δ 5.64 (s, 2H), 6.87 (d, 1H, J=9.4 Hz), 6.95 (m, 2H), 7.30 (m, 1H), 7.42 (d, 1H, J=9.2 Hz), 8.23 (d of d, 1H, J=10 Hz and 2 Hz), 8.26 (s, 1H), 8.72 (d, 1H, J=2 Hz); MS: 272 (M+H)⁺; HPLC Ret Time: 6.99 min (YMC ODS-A 3 um, 4.6×50 mm column, 10 min gradient, 2.5 mL/min).

### F. Preparation of 1-(3-Fluoro-benzyl)-1H-indazol-5-ylamine (Compound C)

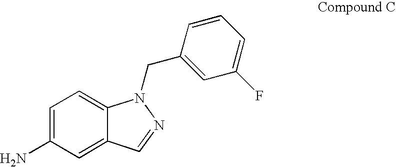

Benzyl nitro indazole (1 equiv.) was charged to a hydogenator, THF (8 volumes) was added and hydrogenated at 15 psi between 30-40° C. The reaction mixture was held for ˜1 h (s.m. <3% by HPLC) cooled to 25° C., the catalyst was filtered and the mixture was washed with THF (0.9 volumes). The mixture was transferred to another vessel, rinsed again with THF (0.4 volumes) distilled to the desired volume (5.5 volumes) atmospherically, and heptane was added (15 volumes) between 47-60° C. over 1 h. The slurry was cooled over 1.5 h to 18-23° C. The slurry was held for 1 h, filtered and washed with THF/heptane (1:4, 10.4 volumes) and dried in oven <45° C., (LOD<1%), yield was 84%. melting point=130° C. HPLC Ret Time: 9.09 min.

### G. Preparation of 4-[1-(3-Fluoro-benzyl)-1H-indazol-5-ylamino]-5-methyl-pyrrolo[2,1-f][1,2,4]triazine-6-carboxylic acid ethyl ester (19)

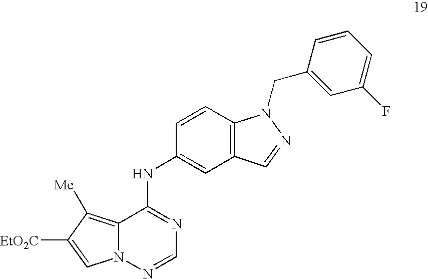

A 3-neck flask was charged with 5-methyl-4-oxo-3,4-dihydr-pyrrolo[2,1-f][1,2,4]triazine-6-carboxylic acid ethyl ester (1.00 equiv.) and dry toluene (15 volumes). POCl₃ (1.2 equiv.) was added in one portion, followed by slow addition of DIEA (1.1 equiv.) at a rate which maintained the temperature below 30° C. The resulting suspension was heated to 111° C. for 24 h becoming homogeneous at 80° C. The reaction was monitored by HPLC after quenching with 2 M MeNH₂/THF (10 μL reaction mixture, 20 μL MeNH₂/THF in 200 μL acetonitrile). Upon completion, the reaction was cooled to −2° C. and was added to a solution of K₂HPO₄ (3.98 equiv) in H₂O (15.6 volumes) while maintaining the temperature below 101C. The solution was stirred for 20 min at −22° C. The resulting light suspension was filtered through a pad of Celite and the layers were separated. The organic layer was washed with 23.5 wt % K₂HPO₄ in H₂O (2.94 volumes), followed by water (2.47 volumes). The solution was filtered and concentrated by heating over the temperature range of 22° C. to 58° C.; until HPLC ratio of toluene to 4-chloro-5-methylpyrrolo[2,1-f][1,2,4]triazine-6-carboxylic acid ethyl ester is 26-36%. The solution was cooled from 58° C. to 40-50° C. To the resulting suspension was added 1-(3-fluoro-benzyl)-1H-indazol-5-ylamine (0.988 equiv) and DIEA (1.1 equiv). The reaction was heated to 70-80° C. and held at this temperature until it was complete by HPLC. It was then cooled to 55° C. and isopropyl alcohol (15.5 volumes) was added. The mixture was cooled from 55° C. to 22° C. over a period of 1.8-2.2 hr. and filtered. The filter cake was washed with cold isopropyl alcohol (2×5.5 volumes) and dried under vacuum <50° C. to afford the product as a cream colored crystalline solid in 84% yield.

¹H NMR (500 MHz, CDCl₃) δ 1.39 (t, 3H, J=7.15 Hz), 2.93 (s, 3H), 4.35 (q, 2H, J=7.15 Hz), 5.59 (s, 2H), 6.86 (d, 1H, J=9.34H), 6.97 (m, 2H), 7.26 (ddd, 1H, J=6.04, 8.24, 14.29 Hz), 7.35 (d, 1H, J=8.80 Hz), 7.42 (br s, 1H), 7.49 (dd, 1H, J=1.65, 8.80 Hz), 7.91 (s, 1H), 8.00 (s, 1H), 8.07 (s, 1H), 8.09 (s, 1H); MS: 445 (M+H)⁺; HPLC Ret Time: 3.847 min (YMC S5 ODS 4.6×50 mm column, 4 min gradient, 3 mL/min).

### H. Preparation of 4-[1-(3-Fluoro-benzyl)-1H-indazol-5-ylamino]-5-methyl-pyrrolo[2,1-j][1,2,4]triazine-6-carboxylic acid (20)

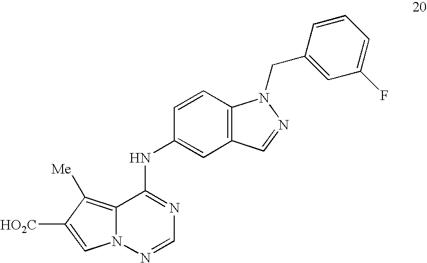

A flask equipped with mechanical stirrer was charged with 4-[1-(3-fluoro-benzyl)-1H-indazol-5-ylamino]-5-methyl-pyrrolo[2,1-f][1,2,4]triazine-6-carboxylic acid ethyl ester (19) (1 equiv), THF (4 volumes) and MeOH (2.5 volumes). The suspension was cooled to 5° C. and 50% NaOH (5.3 equiv.) solution was slowly added maintaining the temperature below 15° C. The resulting solution was warmed to 60° C. for 4 h, and then cooled to 25° C. THF (7 volumes) was charged to the reaction and concentrated HCl (9.95 equiv.) was slowly added maintaining the temperature below 35° C. to pH 3. The resulting slurry was stirred at ambient temperature overnight, and then filtered. The filter cake was washed with H₂O (3×5 volumes) and dried on the filter for 1 h. The filter cake was washed with heptane (1×1 volume) and dried under vacuum at 50° C. to afford the product in 88% yield as an off-white solid.

¹H NMR (500 MHz, DMSO-d₆) δ 2.86 (s, 3H), 5.71 (s, 2H), 7.04 (m, 2H), 7.10 (dd, 1H, J=1.65, 8.80 Hz), 7.17 (d, 1H, J=7.70 Hz), 7.25 (t, 1H, J=7.70 Hz), 7.37 (dd, (1H, J=7.70, 13.74 Hz), 7.57 (dd, 1H, J=1.65, 8.80 Hz), 7.73 (d, 1H, J=8.80 Hz), 7.87 (s, 1H), 8.05 (d, 1H, J=8.35 Hz), 8.16 (s, 1H), 8.83 (s, 1H), 12.47 (s, 1H); MS: 417 (M+H)⁺; HPLC Ret Time: 3.350 min (YMC S5 ODS 4.6×50 mm column, 4 min gradient, 3 mL/min).

### I. Preparation of 3-[[[[[[5-ethyl-4-[[(1-(3-fluorophenyl)methyl)-1H-indazol-5-yl]amino]pyrrolo[2,1-f][1,2,4]triazin-6-yl]amino]carbonyl]oxy]methyl]-4-morpholinecarboxylic acid, (3S)-1,1-dimethylethyl ester (21)

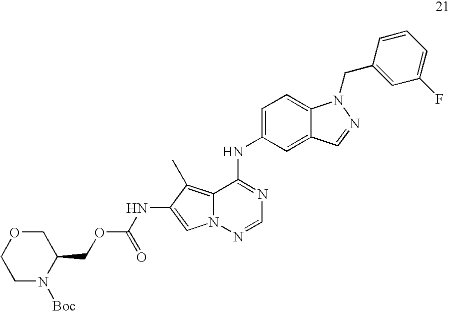

A flask was charged with 4-[1-(3-fluoro-benzyl)-1H-indazol-5-ylamino]-5-methyl-pyrrolo[2,1-f][1,2,4]triazine-6-carboxylic acid (20) (1 equiv.) and toluene (15 volumes). Residual water was removed by azeotropic distillation and the supernatant was analyzed for water content (KF: <200 ppm water). The flask was then charged with 3-hydroxymethyl-morpholine-4-carboxylic acid tert-butyl ester (1.05 equiv.) at about 77° C. Triethyl amine (1.2 equiv) and diphenylphosphoryl azide (1.2 equiv) were added between 77-85° C. The reaction was heated at −87° C. until it was found complete by HPLC. The reaction was cooled to 25° C. diluted with THF (15 volumes) and washed with 10% K₂CO₃ (10 volumes), saturated NaCl (10 volumes) and water (10 volumes) respectively. The product rich organic layer was polish filtered and distilled at atmospheric pressure until the pot temperature was >100° C. The final volume was adjusted to 15 volumes by adding toluene (if necessary). The mixture was cooled to 80° C., water (1 equiv) was added and the product was crystallized. The slurry was cooled to 25° C. over 1 h and held for 17 h. The solid was collected by filtration and the filter cake was rinsed with toluene (2×2 volumes). The solid was air dried overnight and then dried under vacuum at 50° C. to give the product in 82% yield.

¹H NMR (DMSO) δ 1.38 (s, 9H), 2.53 (m, 3H), 3.35-4.34 (m, 10H), 5.71 (s, 2H), 7.03-7.37 (m, 4H), 7.57 (d of d, 1H, J=9 Hz and 1.7 Hz), 7.70 (d, 1H, J=9 Hz), 7.82 (s, 1H), 8.08 (d, 1H, J=1 Hz), 8.15 (s, 1H), 8.58 (s, 1H); MS: 631 (M+H)⁺; HPLC Ret Time: 5.01 min (YMC ODS-A 3 um, 4.6×50 mm column, 10 min gradient, 2.5 mL/min).

### J. Preparation of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester (Ia)

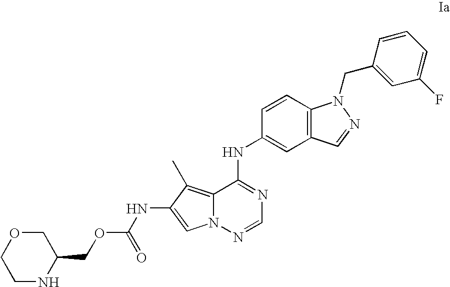

A flask was charged with 3-[[[[[[5-ethyl-4-[[(1-(3-fluorophenyl)methyl)-1H-indazol-5-yl]amino]pyrrolo[2,1-1][1,2,4]triazin-6-yl]amino]carbonyl]oxy]methyl]-4-morpholinecarboxylic acid, (3S)-1,1-dimethylethyl ester (21)(1 equiv.), 7 volumes of water, 1 volume of methanol and concentrated HCl solution (5.0 equivalents). The slurry was heated to 70° C. and held at this temperature until found complete by HPLC. After completion, water (3 volumes) was charged into the hot reaction mixture which cooled the mixture to 45-55° C. The mixture was filtered and the filtrate was extracted with ethyl acetate (2×6 volumes). Ethyl acetate (10 volumes), methanol (2-3 volumes) and BHA (2.7 wt %) was charged into the isolated aqueous phase. Using 50% NaOH solution, the pH of the mixture was adjusted to pH 9-13. The phases were allowed to separate. The product rich organic layer was collected and water (10 volumes) was added into the mixture at 55-60° C. in 15-30 min. The mixture was held at 55-60° C. for 30 min after addition of water, then cooled to 19-25° C. over 1 h. The product was filtered and washed with ethyl acetate (2×3 volumes). The filter cake was reslurried with ethyl acetate (15 volumes) and BHA (2.7 wt %) was added. The resulting slurry was distilled at atmospheric pressure to remove moisture. The volume of the mixture was adjusted to 8-10 volumes while maintaining the batch temperature at 74-78° C. The mixture was cooled to 19-25° C. over an hour. The solid was collected by filtration and the filter cake was rinsed with ethyl acetate (2.2 volumes). The solid was dried under vacuum at 45° C. to afford a crystalline solid (Form N-2) in 77% yield (HPLC AP 99.2).

¹H NMR (DMSO) δ 2.51 (m, 1H), 2.57 (s, 3H), 3.10-4.04 (m, 10H), 4.35 (m, 2H), 5.71 (s, 2H), 7.03-7.13 (m, 3H), 7.37 (m, 1H), 7.59 (m, 1H), 7.71 (m, 1H), 7.83 (s, 2H), 8.07 (s, 1H), 8.15 (s, 1H), 8.61 (s, 1H), 9.47 (s, 1H), 9.87 (s, 1H); MS: 531 (M+H)⁺; HPLC Ret Time: 4.55 min (YMC ODS-A 3 um, 4.6×50 mm column, 10 min gradient, 2.5 mL/min).

Additional compounds that can be prepared by the process of the invention include those shown in Table 1 wherein R, R¹ and R² are as shown.

TABLE 1

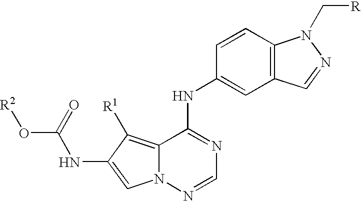

| | | | | | HPLC |
|---|---|---|---|---|---|
| | | | | | Ret Time |
| R | R¹ | R² | Compound Name | [M + ] | (min) |
| 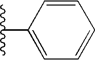 | ethyl | 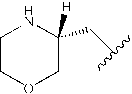 | [5-ethyl-4-[[(1- phenylmethyl)-1H- indazol-5- yl]amino]pyrrolo[2,1- f][1,2,4]triazin-6-yl]- carbamic acid, (3S)-3- morpholinylmethyl ester, monohydrochloride | 527 | 9.95 |
| 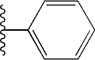 | ethyl | 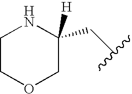 | [5-ethyl-4-[[(1- phenylmethyl)-1H- indazol-5- yl]amino]pyrrolo[2,1- f][1,2,4]triazin-6-yl]- carbamic acid, (3R)-3- morpholinylmethyl ester | 527 | 10.06 |
| 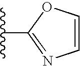 | ethyl | 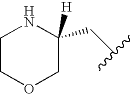 | [5-ethyl-4-[[1-(2- oxazolylmethyl)-1H- indazol-5- yl]amino]pyrrolo[2,1- f][1,2,4]triazin-6-yl]- carbamic acid, (3S)-3- morpholinylmethyl ester | 518 | 6.70 |
| 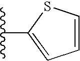 | ethyl | 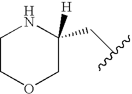 | [5-ethyl-4-[[1-(2- thienylmethyl)-1H- indazol-5- yl]amino]pyrrolo[2,1- f][1,2,4]triazin-6-yl]- carbamic acid, (3S)-3- morpholinylmethyl ester | 533 | 9.70 |
| 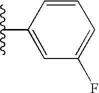 | ethyl | 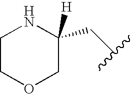 | [5-ethyl-4-[[1-[(3- fluorophenyl)methyl]-1H- indazol-5- yl]amino]pyrrolo[2,1- f][1,2,4]triazin-6-yl]- carbamic acid, (3S)-3- morpholinylmethyl ester | 545 | 10.21 |
| 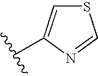 | ethyl | 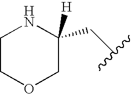 | [5-ethyl-4-[[1-(4- thiazolylmethyl)-1H- indazol-5- yl]amino]pyrrolo[2,1- f][1,2,4]triazin-6-yl]- carbamic acid, (3S)-3- morpholinylmethyl ester | 534 | 7.98 |
| 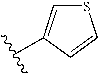 | ethyl | 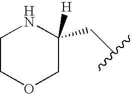 | [5-ethyl-4-[[1-(3- thienylmethyl)-1H- indazol-5- yl]amino]pyrrolo[2,1- f][1,2,4]triazin-6-yl]- carbamic acid, (3S)-3- morpholinylmethyl ester | 533 | 9.68 |
| 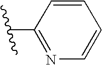 | ethyl | 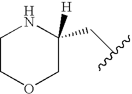 | [5-ethyl-4-[[1-(2- pyridinylmethyl)-1H- indazol-5- yl]amino]pyrrolo[2,1- f][1,2,4]triazin-6-yl]- carbamic acid, (3S)-3- morpholinylmethyl ester | 528 | 7.14 |
| 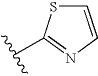 | ethyl | 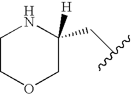 | [5-ethyl-4-[[1-(2- thiazolylmethyl)-1H- indazol-5- yl]amino]pyrrolo[2,1- f][1,2,4]triazin-6-yl]- carbamic acid, (3S)-3- morpholinylmethyl ester | 534 | 8.21 |
| 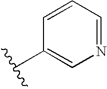 | ethyl | 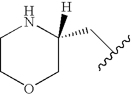 | [5-ethyl-4-[[1-(3- pyridinylmethyl)-1H- indazol-5- yl]amino]pyrrolo[2,1- f][1,2,4]triazin-6-yl]- carbamic acid, (3S)-3- morpholinylmethyl ester | 528 | 6.74 |
| 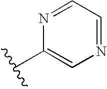 | ethyl | 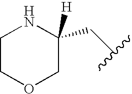 | [5-ethyl-4-[[1- (pyrazinylmethyl)-1H- indazol-5- yl]amino]pyrrolo[2,1- f][1,2,4]triazin-6-yl]- carbamic acid, (3S)-3- morpholinylmethyl ester | 529 | 7.46 |
| 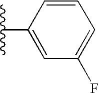 | methyl | 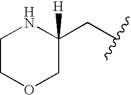 | [4-[[1-(3- fluorophenyl)methyl]-1H- indazol-5-ylamino]-5- methyl-pyrrolo[2,1- f][1,2,4]triazin-6-yl]- carbamic acid, (3S)-3- morpholinylmethyl ester | 531 | 2.48² |
| 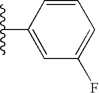 | methyl | 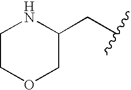 | [4-[[1-(3- fluorophenyl)methyl]-1H- indazol-5-ylamino]-5- methyl-pyrrolo[2,1- f][1,2,4]triazin-6-yl]- carbamic acid, 3- morpholinylmethyl ester | 531 | 1.97² |
| 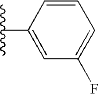 | methyl | 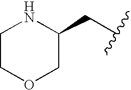 | [4-[[1-(3- fluorophenyl)methyl]-1H- indazol-5-ylamino]-5- methyl-pyrrolo[2,1- f][1,2,4]triazin-6-yl]- carbamic acid, (3R)-3- morpholinylmethyl ester | 531 | 1.97² |

### EXAMPLE 2

### Preparation of Monohydrate Crystalline Form H-1 of the Compound Ia

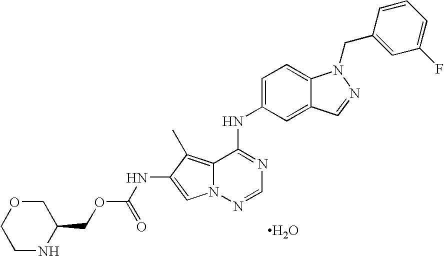

A 1-L flask was charged with 3-[[[[[5-ethyl-4-[[(1-(3-fluorophenyl)methyl)-1H-indazol-5-yl]amino]pyrrolo[2,1-f][1,2,4]triazin-6-yl]amino]carbonyl]oxy]methyl]-4-morpholinecarboxylic acid, (3S)-1,1-dimethylethyl ester (39.8 g, 63.2 mmol) and methanol (300 mL). To the suspension was added concentrated HCl (26 mL, 316 mmol) over 15 min (max. temperature reached 30° C.). The resulting solution was stirred at 55° C. for 2 h. The reaction was cooled to 25° C. and diluted with DM water (600 mL). The resulting solution was filtered through #5 paper to remove fine particles. The solution was transferred into 2-L separatory funnel. Ethyl acetate (500 mL) was added and the contents of the funnel were stirred for 5 min. The phases were allowed to separate. The product rich bottom layer was collected and washed with additional ethyl acetate (300 mL) as described above. The product rich bottom layer was charged into 2-L flask. Ethyl acetate (300 mL) was added and stirred (pH=1.3). Using 50% NaOH solution (˜25 mL), pH of the mixture was adjusted to pH ˜10. The mixture was transferred into 2-L separatory funnel. The phases were allowed to separate. The product rich organic layer was collected. The aqueous layer was extracted with ethyl acetate (300 mL). Combined product rich organic extracts were dried with MgSO₄. The MgSO₄ was removed by filtering. The filtrate was concentrated in vacuo to a tan solid to yield 31.8 g of Compound Ia.

Elemental analysis:

% Calc.: % C, 59.17; % H, 5.32; % N, 20.45.

% Found: % C, 58.94; % H, 5.31; % N, 20.07.

KF Moisture: 3.18% (0.97 moles).

### Preparation of Monohydrate Crystalline Form H-1 of the Compound Ia

### Alternate Procedure

A flask was charged with 3-[[[[[5-ethyl-4-[[(1-(3-fluorophenyl)methyl)-1H-indazol-5-yl]amino]pyrrolo[2,1-f][1,2,4]triazin-6-yl]amino]carbonyl]oxy]methyl]-4-morpholinecarboxylic acid, (3S)-1,1-dimethylethyl ester (1 equiv.), 7 volumes of water, 1 volume of methanol, and concentrated HCl solution (5.0 equivalents). The slurry was heated to 70° C. and held at this temperature until the reaction was found to be complete by HPLC. After completion, water (3 volumes) was charged into the hot reaction mixture which cooled the mixture to 45-55° C. The mixture was filtered and the filtrate was extracted with ethyl acetate (2×6 volumes). Ethyl acetate (10 volumes) and BHA (2.7 wt %) was charged into the isolated aqueous phase. Using 25% NaOH solution, the pH of the mixture was adjusted to pH 9-13. This mixture was held at 19-25° C. for 2 h. The crystallized product was filtered from the mixture and sequentially washed with water (4 volumes) and ethyl acetate (4 volumes). The monohydrate was obtained as a white crystalline solid (HPLC 99.2 AP) after air drying the wet cake.

### EXAMPLE 3

### Preparation of the N-1 Crystalline Form Compound Ib

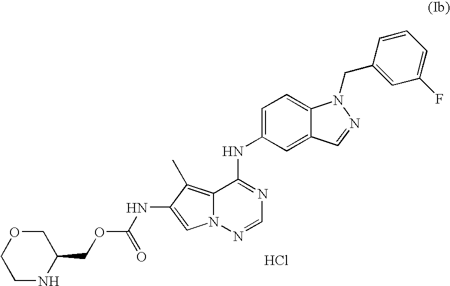

Compound Ib is the hydrochloric acid salt of Compound Ia.

A 5-L flask was charged with 3-[[[[[5-ethyl-4-[[(1-(3-fluorophenyl)methyl)-1H-indazol-5-yl]amino]pyrrolo[2,1-f][1,2,4]triazin-6-yl]amino]carbonyl]oxy]methyl]-4-morpholinecarboxylic acid, (3S)-1,1-dimethylethyl ester (330 g, 0.51 mol) and methanol (2.5 L). To the suspension was added concentrated HCl (170 mL, 2.04 mol) over 15 min (max. temperature reached 30° C.). The resulting solution was stirred at 55° C. for 2 h. The reaction was cooled to 25° C. and diluted with DM water (5 L). The resulting solution was filtered through #5 paper to remove fine particles. The solution was transferred into 10-L vessel. Ethyl acetate (5 L) was added and stirred for 5 min. The phases were allowed to separate. The product rich bottom layer was collected and washed with additional ethyl acetate (2 L) as described above. The product rich bottom layer was charged back into 10-L reactor. Ethyl acetate (2.5 L) was added and stirred (pH=1.3). Using 50% NaOH solution (about 190 mL), the pH of the mixture was adjusted to pH 9.5-10. The phases were allowed to separate. The product rich organic layer was collected. The aqueous layer was extracted with ethyl acetate (2.5 L). Combined product rich organic extracts were filtered (through #5 paper). The filtrate was concentrated in vacuo to a solid. Water was decanted from the solid. The solid was transferred into 10-L reactor using ethyl acetate (2 L) and methanol (2 L). The resulting suspension was heated to 50° C. to obtain a homogeneous solution. Concentrated HCl (41 mL, 0.49 mol) was added slowly over 15 min. Solid crystallized from the solution and formed a slurry. The slurry was cooled to 25° C. over 1 h. The solid was collected by filtration and the filter cake was rinsed with 1:1 ethyl acetate:methanol (1×500 mL) and with ethyl acetate (1×500 mL). The crystalline solid was air dried for 1 h and then dried under vacuum at 45° C. to yield 204 g of Compound Ib, the hydrochloric acid salt of Compound Ia. (HPLC AP 99.6).

¹H NMR (DMSO) δ 2.51 (m, 1H), 2.57 (s, 3H), 3.10-4.04 (m, 10H), 4.35 (m, 2H), 5.71 (s, 2H), 7.03-7.13 (m, 3H), 7.37 (m, 1H), 7.59 (m, 1H), 7.71 (m, 1H), 7.83 (s, 2H), 8.07 (s, 1H), 8.15 (s, 1H), 8.61 (s, 1H), 9.47 (s, 1H), 9.87 (s, 1H); MS: 531 (M+H)⁺; HPLC Ret Time: 4.55 min (YMC ODS-A 3 um, 4.6×50 mm column, 10 min gradient, 2.5 mL/min).

### EXAMPLE 4

### Crystalline Forms of [4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester (Ia)

The crystalline forms prepared in Examples 1 to 3 were characterized by x-ray and other techniques. The unit cell parameters are tabulated in Table 2. The unit cell parameters were obtained from single crystal X-ray crystallographic analysis.

TABLE 2
Unit Cell Parameters and Melting Points

| Parameter | N-2 | H-1 | N-1 |
|---|---|---|---|
| a (Å) | 10.16 | 8.78 | 5.32 |
| b (Å) | 10.46 | 10.78 | 10.92 |
| c (Å) | 12.48 | 14.08 | 22.95 |
| α (degrees) | 96.4 | 99.6 | 90.0 |
| β (degrees) | 103.3 | 95.8 | 94.9 |
| γ (degrees) | 93.7 | 93.3 | 90.0 |
| Space Group | P1 | P1 | P2₁ |
| Molecules/unit cell* | 2 | 2 | 2 |
| Volume (Å³) | 1277.5 | 1303.9 | 1327.6 |
| Density (calculated) (g/cm³) | 1.379 | 1.397 | 1.418 |
| Temperature (° C.) | 25 | 25 | 25 |
| Melting Point (° C.) | 166-174 | 116-136 | 207-240 |

*Molecules/unit cell represent the number of molecules of Compound Ia per unit cell.

TABLE 3
Several Peaks (2θ values) from Powder X-Ray Diffraction
Patterns (CuKα λ = 1.5418 Å)

| Form | Diffraction Peak Positions (degrees 2θ ± 0.1) at 22° C. | | | | | | | |
|---|---|---|---|---|---|---|---|---|
| N-2 | 7.3 | 8.6 | 12.0 | 17.8 | 19.3 | 20.1 | 25.6 | — |
| H-1 | 6.5 | 10.2 | 11.4 | 15.5 | 18.3 | 22.9 | 25.8 | 28.4 |
| N-1 | 3.9 | 9.0 | 11.3 | 14.2 | 16.8 | 25.3 | 26.9 | — |

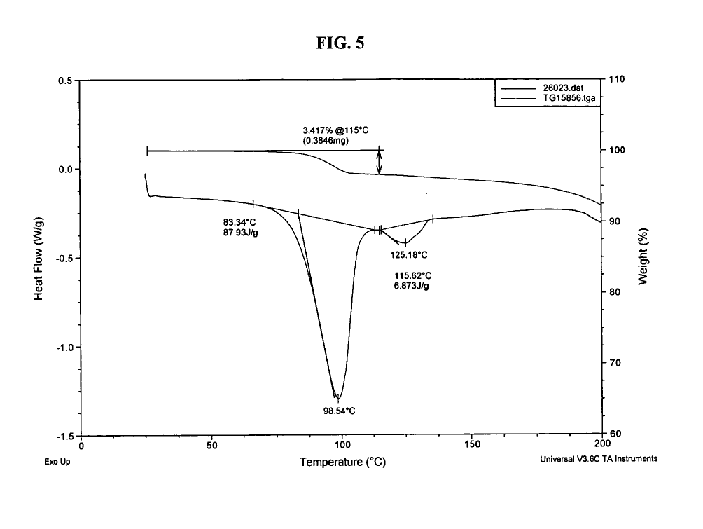

FIG. 5 shows the thermogravimetric weight loss for the monohydrate form (H-1) of Compound Ia. The H-1 form exhibited dehydration weight loss of approximately 3.4 weight % at a temperature of 115° C. Theoretical weight loss of water from the monohydrate form H-1 is 3.5 weight %.

What is claimed is:

1. A crystalline form of Compound Ia: 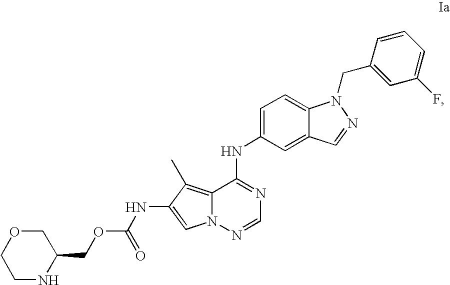
comprising Form N-2.

2. The crystalline form according to claim 1 consisting essentially of Form N-2.

3. The crystalline form according to claim 2, wherein said Form N-2 is in substantially pure form.

4. The crystalline form according to claim 1 characterized by unit cell parameters substantially equal to the following:
Cell dimensions: a=10.16 Å
b=10.46 Å
c=12.48 Å
α=96.4 degrees
β=103.3 degrees
γ=93.7 degrees
Space group: P1
Molecules/unit cell: 2
wherein measurement of said crystalline form is at a temperature of about 25° C.

5. The crystalline form according to claim 1 characterized by a powder x-ray diffraction pattern comprising four or more 2θ values (CuKαλ=1.5418 Å) selected from the group consisting of 7.3, 8.6, 12.0, 17.8, 19.3, 20.1, and 25.6, at a temperature of about 22° C.

6. The crystalline form according to claim 5 further characterized by a powder x-ray diffraction pattern comprising five or more 2θ values (CuKαλ=1.5418 Å) selected from the group consisting of 7.3, 8.6, 12.0, 17.8, 19.3, 20.1, and 25.6, at a temperature of about 22° C.

7. The crystalline form according to claim 1 characterized by one or more of the following:
a) a unit cell parameters substantially equal to the following:
Cell dimensions: a=10.16 Å
b=10.46 Å
c=12.48 Å
α=96.4 degrees
β=103.3 degrees
γ=93.7 degrees
Space group: P1
Molecules/unit cell: 2
wherein measurement of said crystalline form is at a temperature of about 25° C.;
b) a powder x-ray diffraction pattern comprising four or more 2θ values (CuKαλ=1.5418 Å) selected from the group consisting of 7.3, 8.6, 12.0, 17.8, 19.3, 20.1, and 25.6, at a temperature of about 22° C.; and/or
c) a melting point in the range of from 166° C. to 174° C.

8. A pharmaceutical composition comprising the crystalline form according to claim 1 and a pharmaceutically acceptable carrier or diluent.

9. The pharmaceutical composition according to claim 8 wherein said Form N−1 is in substantially pure form.

10. A method of treating a proliferative disease, comprising administering to a warm blooded animal in need thereof, a therapeutically-effective amount of the crystalline form of claim 1.

11. The method according to claim 10 wherein said crystalline form is in substantially pure form.

12. A composition comprising Compound Ia: 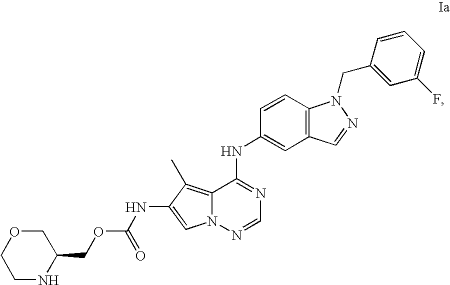
wherein at least 90 weight % of said Compound Ia is in the N-2 crystalline form, based on weight of Compound Ia in said composition.

13. A crystalline form of Compound Ia: 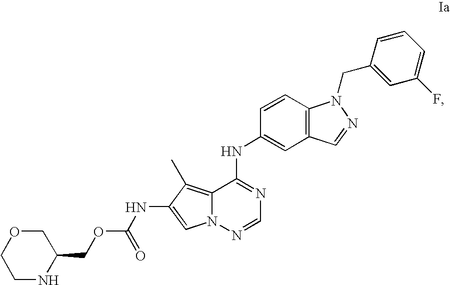
comprising form H-1 monohydrate.

14. The crystalline form according to claim 13 consisting essentially of Form H-1 monohydrate.

15. The crystalline form according to claim 14, wherein said Form H-1 monohydrate is in substantially pure form.

16. The crystalline form according to claim 13 characterized by unit cell parameters substantially equal to the following:
Cell dimensions: a=8.78 Å
b=10.78 Å
c=14.08 Å
α=99.6 degrees
β=95.8 degrees
γ=93.3 degrees
Space group: P1
Molecules/unit cell: 2
wherein measurement of said crystalline form is at a temperature of about 25° C.

17. The crystalline form according to claim 13 characterized by a powder x-ray diffraction pattern comprising four or more 2θ values (CuKαλ=1.5418 Å) selected from the group consisting of 6.5, 10.2, 11.4, 15.5, 18.3, 22.9, 25.8, and 28.4, at a temperature of about 22° C.

18. The crystalline form according to claim 17 further characterized by a powder x-ray diffraction pattern comprising five or more 2θ values (CuKαλ=1.5418 Å) selected from the group consisting of 6.5, 10.2, 11.4, 15.5, 18.3, 22.9, 25.8, and 28.4, at a temperature of about 22° C.

19. A crystalline form of hydrochloric acid salt of Compound Ia: 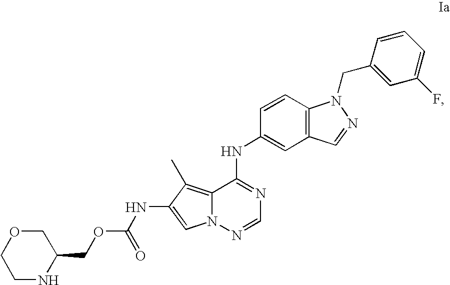
comprising Form N-1.

20. The crystalline form according to claim 19 consisting essentially of Form N-1.

21. The crystalline form according to claim 20, wherein said Form N-1 is in substantially pure form.

22. The crystalline form according to claim 19 characterized by unit cell parameters substantially equal to the following:
Cell dimensions: a=5.32 Å
b=10.92 Å
c=22.95 Å
α=90.0 degrees
β=94.9 degrees
γ=90.0 degrees
Space group: P2₁
Molecules/unit cell: 2
wherein measurement of said crystalline form is at a temperature of about 25° C.

23. The crystalline form according to claim 19 characterized by a powder x-ray diffraction pattern comprising four or more 2θ values (CuKαλ=1.5418 Å) selected from the group consisting of 3.9, 9.0, 11.3, 14.2, 16.8, 25.3, and 26.9, at a temperature of about 22° C.

24. The crystalline form according to claim 23 further characterized by a powder x-ray diffraction pattern comprising five or more 2θ values (CuKαλ=1.5418 Å) selected from the group consisting of 3.9, 9.0, 11.3, 14.2, 16.8, 25.3, and 26.9, at a temperature of about 22° C.
